(19) United States
(12) Patent Application Publication
(10) Pub. No.: US 2012/0094338 A1
(43) Pub. Date: Apr. 19, 2012

(54) CONTINUOUS PRODUCTION AND EXCRETION OF WAXY PRODUCTS FROM PHOTOSYNTHETIC ORGANISMS

(76) Inventors: Zhong Kui Li, University Park, PA (US); Adam Schindler, Longview, TX (US); Jerry Brand, Austin, TX (US); Norman M. Whitton, Houston, TX (US)

(21) Appl. No.: 13/055,458

(22) PCT Filed: Jul. 22, 2009
(86) PCT No.: PCT/US09/51410

§ 371 Date: Dec. 28, 2011

Related U.S. Application Data
(60) Provisional application No. 61/082,750, filed on Jul. 22, 2008.

(51) Int. Cl. C12P 7/64; C12P 1/00

(52) U.S. Cl. 435/134; 435/41

(57) ABSTRACT

This invention disclosed herein relates generally to production and maintenance of genetically modified organisms that are capable of continuously making and excreting valuable waxy products.

### PRIORITY STATEMENT

This application claims priority to U.S. Provisional Patent Application No. 61/082,750, which was filed on Jul. 22, 2008, herein incorporated by reference in its entirety.

### FIELD OF THE INVENTION

This invention relates generally to the field of waxy product production. More specifically, the invention relates to the production of genetically modified organisms that are capable of making and excreting waxy products.

### BACKGROUND

The depletion of the earth's fossil energy resources is one of the major challenges facing today's society. Due to large-scale carbon dioxide emissions, the combustion of the fossil energy materials is one of the key factors responsible for global warming. Thus, alternative energy sources based on sustainable, regenerative, and ecologically friendly processes are urgently needed. One of the most prominent alternative energy resources is biodiesel. As a possible substitute for petroleum-based diesel fuel, a number of countries are already producing biodiesel on a large scale. For example, in 2004, Germany recently produced over 1,080,000 tons of biodiesel. (Bockey & von Schenck, 2005). Biodiesel offers a number of interesting and attractive beneficial properties compared to conventional petroleum-based diesel (for an overview, see Krawczyk, 1996). Since it is based on renewable biological materials, the use of biodiesel contributes to a balanced carbon dioxide cycle. Other benefits arising from the use of biodiesel include, but are not limited to, a reduction in emissions during combustion (e.g., carbon monoxide, sulphur, aromatic hydrocarbons, soot particles). Biodiesel is also non-toxic and completely biodegradable. Because biodiesel also has a high flash point, it has low flammability, and thus the use of biodiesel is very safe and non-hazardous. Furthermore, because biodiesel has good lubrication properties, biodiesel reduces engine wear and tear. With little or no modification, conventional diesel engines can use both pure biodiesel and biodiesel mixed in any ratio with petroleum-based diesel.

Despite positive ecological aspects and the promise of biodiesel, there are numerous drawbacks and limitations to the production of biodiesel on a technical scale. First, biodiesel production is dependent on the availability of sufficient vegetable oil feed stocks. More specifically, these feedstocks include mostly rapeseed in Continental Europe, soybean in North America, and palm oil in South East Asia. Therefore, industrial-scale biodiesel production will remain geographically and seasonally restricted to oilseed producing areas.

Therefore, one of the major limitations impeding a more widespread use of biodiesel is the extensive acreage needed for production of oilseed crops. The yield of biodiesel from rapeseed is only 1300 l ha⁻¹, since only the seed oil is used for biodiesel production. The other, major part of the plant biomass is not used for this purpose. Furthermore, oilseed crops like rapeseed and soybean are not self-compatible; therefore, their cultivation requires a frequent crop-rotation regime. Consequently, in the future, biodiesel based on oilseed crops will probably not be able to substitute more than 5-15% of petroleum-based diesel.

Other problems include the reliability of product quality when produced in bulk, and the clogging of filters at low temperatures due to crystallization. Therefore, plant oils must be transesterified with short chain alcohols (like methanol or ethanol) to yield the FAME and FAEE constituents of biodiesel. This transesterification process and the subsequent purification steps are cost intensive and energy consuming, and thereby reduce the possible energy yield and increase the price. FAMEs and FAEEs have comparable chemical and physical fuel properties and engine performances (Peterson et al., 1995), but for economic reasons, only FAMEs are currently produced on an industrial scale. Currently, methanol is much cheaper than ethanol. But, methanol is mainly produced from natural gas. Thus, FAME-based biodiesel is not a truly renewable product since the alcohol component is of fossil origin. Furthermore, methanol is highly toxic and hazardous, and its use requires special precautions. Use of bioethanol for production of FAEE-based biodiesel would result in a fully sustainable fuel, but only at the expense of much higher production costs. (Kalscheuer et al., 2006).

Linear wax esters are lipophilic compounds containing a long chain fatty alcohol that is esterified to a long chain fatty acid. These wax esters are found in a number of diverse organisms ranging from mammals to plants to bacteria. Wax esters have a multitude of important commercial applications in a variety of technical areas, including but not limited to the medical, cosmetics, and food industries. More specifically, wax esters are useful for several cosmetic applications including (1) as a lubricant, (2) as an additive for leather processing, (3) as a carrier for pharmaceuticals, and (4) as a solvent. Hydrogenation of the wax eliminates the double bonds and produces a hard wax that is useful for surface treatments. This hard wax is useful in textile sizing, in coating paper containers, and in cosmetics (e.g., lipstick and creams). Sulphurization of the wax or other modifications make the substance useful in specialty lubricant applications, as a textile softener, as a component of printing inks, and as a component in many technical products such as corrosion inhibitors, surfactants, detergents, disinfectants, plasticizers, resins, and emulsifiers.

The most detailed information concerning wax ester biosynthesis concerns wax biosynthesis in jojoba plants, where it appears that two enzymes catalyze the formation of wax esters. The first step of the pathway is catalyzed by a fatty acyl-CoA reductase that reduces very long chain fatty acyl CoA (a very long chain fatty acyl CoA generally having greater than 18 carbons). This reductase catalyzes the formation of a long chain alcohol directly from this substrate via an aldehyde intermediate. The second enzyme, a wax ester synthase, is an acyl-CoA-fatty alcohol transferase that catalyzes the formation of an ester linkage between acyl-CoA and a fatty alcohol to yield a wax ester.

Unlike the pathway in jojoba plant, the pathway of wax ester biosynthesis in A. calcoaceticus comprises three enzymatic steps. First, acyl-CoA is reduced to the corresponding fatty aldehyde by an NADPH-dependent acyl-CoA reductase. Second, the aldehyde is further reduced to the corresponding fatty alcohol catalyzed by the fatty aldehyde reductase. Third, acyl-CoA:fatty alcohol acyl transferase (wax ester synthase) condenses the fatty alcohol with acyl-CoA, which results in the formation of the wax ester.

Accordingly, one of the key enzymatic steps involved in wax ester biosynthesis is the transfer of an acyl chain from fatty acyl-CoA to a fatty alcohol. This reaction is catalyzed by wax ester synthase. While several wax ester synthases have been described in terms of their substrate specificities and intracellular locations, very little is known about the proteins associated with this activity and the genes encoding this enzyme. For example, the atfA enzyme from Acinetobacter baylyi (formerly known as Acinetobacter sp. strain ADP1) is a highly non-specific wax synthase, and it will transesterify a number of different substrates including (1) short chain to very long chain-length linear primary alkyl alcohols, (2) cyclic, phenolic, and secondary alcohols, (3) diols and dithiols, and (4) mono- and diacylglycerols as well as sterols. (Kalscheuer et al., 2006). Conversely, human acyl-CoA:ethanol acyltransferase enzyme exhibits a high specificity for both ethanol and long chain fatty acyl-CoA molecules.

A number of alternatives for the production of ethanol from living organisms have been investigated using microorganisms. The production of ethanol by microorganisms has, in large part, been investigated using the yeast Saccharomyces and bacteria Zymomonas. Both of these microorganisms contain the genetic information to produce enzymes pyruvate decarboxylase (PDC) and alcohol dehydrogenase (ADH), which are used to produce ethanol from pyruvate, a product of the glycolytic pathway. U.S. Pat. No. 4,242,455 to Muller et al. describes a continuous process in which an aqueous slurry of carbohydrate polymer particles, such as starch granules and/or cellulose chips, fibres, etc., are acidified with a strong inorganic acid to form a fermentable sugar. The fermentable sugar is then fermented to ethanol with at least two strains of Saccaromyces. U.S. Pat. No. 4,350,765 to Chibata et al. describes a method of producing ethanol in a high concentration by using an immobilized Saccharomyces or Zymomonas and a nutrient culture broth containing a fermentative sugar. U.S. Pat. No. 4,413,058 to Arcuri et al. describes a new strain of Zymomonas mobilis that is used to produce ethanol by placing the microorganism in a continuous reactor column and passing a stream of aqueous sugar through said column. PCT Application WO/88/09379 to Hartley et al. describes the use of facultative anaerobic thermophilic bacteria strains that produce ethanol by fermenting a wide range of sugars, including cellobiose and pentoses. These bacteria strains contain a mutation in lactate dehydrogenase. As a result, rather than produce lactate under anaerobic conditions, these microorganisms produce ethanol instead.

In addition, Escherichia coli has been genetically altered to produce ethanol by inserting the genetic material encoding for AHD B and PDC. Even this altered Escherichia coli requires a variety of organic substrates for bacterial metabolism and growth. (Ingram et al., 1987).

All of the above prior art describe microorganisms which utilize a carbohydrate/sugar substrate to produce ethanol. As such, these processes are costly because it is necessary to provide the microorganisms with carbohydrates and sugars. Hence, the cost of these systems is a deterrent to the refinement and scale up of such systems for the production of ethanol.

It is highly desirable to find a microorganism which can effectively produce ethanol wherein said microorganism requires minimal feed substrate. There are some rare cases, such as the green alga Neochloris oleoabundans, which do grow reasonably quickly and can produce high amounts of lipid as a percentage of their dry weight. But, even in this case, the bulk culture must be concentrated and dried. Then, the hydrophobic fraction must be chemically extracted. Using exogenous ethanol, this lipid fraction is further processed to produce a usable biofuel. Even if a photobioreactor can cheaply produce a large amount of lipid contained in the algae in bulk culture, the cost of converting this widely dispersed and contaminated material into a chemical reagent and then into a fuel product renders the process uncompetitive with petroleum based fuels.

Even in an ideal chemostatic photobioreactor, in which an expensive and elaborate system maintains an algal culture at its optimal cell concentration via constant dilution, the culture conditions that are required for rapid growth prevent lipid accumulation. Hence, most algae do not naturally accumulate lipids, and even those that do rarely allow the lipid fraction to exceed 30% of the cell's dry weight. Instead, algae maintain a small pool of ready fatty acids called fatty acyl-CoA, which are used as needed for membrane and cell component synthesis. An elaborate and poorly understood control mechanism keeps this fatty acyl-CoA pool at a steady state, increasing synthesis when the pool is depleted and slowing synthesis when the pool is “full”. A system that synthesizes a foreign lipid molecule (FAEEs) and excretes it effectively removes that molecule from equilibrium of metabolites that the cell tightly controls. In effect, the cell's metabolism still works to keep a small pool of fatty-acyl CoA molecules ready for use, and it will self-adjust to the continuous disruption of this equilibrium by the exogenous genes.

Hence, it is highly desirable to generate a microorganism that can produce sufficient quantities of alcohol in vivo for the subsequent generation and excretion of these valuable waxy products.

### SUMMARY OF THE INVENTION

Disclosed herein are methods and compositions relating to the genetic modification of a cell for the in vivo production of ethanol, and the subsequent generation and excretion of FAEEs. In particular, this invention relates to the genetic modification of microorganisms, which modification incorporates the genetic information comprising pyruvate acyl-coA:ethanol acyltransferase (AEAT), pyruvate decarboxylase (PDC), and alcohol dehydrogenase (ADH).

Also disclosed are cells comprising at least one transgenic insert comprising acyl-CoA:ethanol acyltransferase (AEAT).

Further disclosed is a nucleic acid comprising the nucleotide sequences in SEQ ID NO. 1 and SEQ ID NO. 2.

Disclosed herein are methods of producing FAEEs in a cell comprising introducing into the cell at least one transgenic insert comprising acyl-CoA:ethanol acyltransferase (AEAT), wherein the cell is capable of producing FAEEs.

Also disclosed are methods of excreting a product from a cell, wherein the cell comprises a transmembrane pump, the method comprising producing a product in the cell, wherein the product is excreted from the cell into the medium via the transmembrane pump.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic of the plasmid pAQ-EX1.

FIG. 2 shows a schematic of 3 genes (pdc, adhB, and atfA) in a plasmid.

FIG. 3 shows a schematic of 3 genes (pdc, adhB, and AEAT) in a plasmid

FIG. 4 shows a schematic of the same 3 genes (pdc, adhB, and AEAT) as shown in FIG. 3, and shows that more than 1 transgenic insert for each gene can be added (n=number of transgene inserts for each gene).

FIG. 5 shows a schematic of 4 genes (pdc, adhB, AEAT, and RND) in a plasmid.

FIG. 6 shows a schematic of the same 5 genes (pdc, adhB, AEAT, and RND) as shown in FIG. 5, and shows that more than 1 transgenic insert for each gene can be added (n=number of transgene inserts for each gene).

### DETAILED DESCRIPTION

Before the present compounds, compositions, and methods are disclosed and described, it is to be understood that they are not limited to specific methods unless otherwise specified, or to particular reagents unless otherwise specified, as such may, of course, vary. It is also to be understood that the terminology used herein is for the purpose of describing particular aspects only and is not intended to be limiting. The present application also individually and specifically incorporates by reference into this disclosure the publications for the material that is discussed in the sentence in which the publication is relied upon.

### 1. DEFINITIONS

In this specification and in the claims that follow, reference will be made to a number of terms which shall be defined to have the following meanings:

Unless the context clearly dictates otherwise, the singular forms “a,” “an,” and “the” include plural referents.

Ranges can be expressed herein as from “about” one particular value, and/or to “about” another particular value. When such a range is expressed, another aspect includes—from the one particular value and/or to the other particular value. Similarly, when values are expressed as approximations, by use of the antecedent “about,” it will be understood that the particular value forms another aspect. It will be further understood that each endpoints is significant in relation to the other endpoint, and is significant independent of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “amino acid” is used in its broadest sense, and includes naturally occurring amino acids as well as non-naturally occurring amino acids, including amino acid analogs and derivatives. The latter includes molecules containing an amino acid moiety.

The term “biodiesel” refers to an alternative energy source and a substitute for petroleum-based diesel fuel.

The term “biological function” or “biological activity” refers to the ability of a wax ester synthase protein to catalyze the transfer of an acyl chain from fatty acyl-CoA to fatty alcohol via condensation of the fatty alcohol with the acyl-CoA, thereby resulting in the formation of a wax ester.

The term “cell” refers to the basic structural and functional unit that makes up all living organisms. Typically, it is the smallest unit of an organism that is classified as living. Two types of cells are commonly recognized—eukaryotic cells and prokaryotic cells.

The term “cyanobacteria” refers to a specific type of cell. Autotrophic cyanobacteria were once classified as “blue green algae” because of their superficial resemblance to eukaryotic green algae. Although both groups are photosynthetic, they are only distantly related: cyanobacteria lack internal organelles, a discrete nucleus, and the histone proteins associated with eukaryotic chromosomes. Like all eubacteria, their cell walls contain peptidoglycan. Studies of metabolic similarities and ribosomal RNA sequence suggest that cyanobacteria form a good, monophyletic taxon. Because motile species of cyanobacteria utilize the same mysterious gliding locomotion as the gram-negative gliding bacteria, some microbiologists suggest that cyanobacteria should be classified together as a subgroup of gliding bacteria. Although they are truly prokaryotic, cyanobacteria have an elaborate and highly organized system of internal membranes which function in photosynthesis. Chlorophyll a and several accessory pigments (phycoerythrin and phycocyanin) are embedded in these photosynthetic lamellae, the analogs of the eukaryotic thylakoid membranes. The photosynthetic pigments impart a rainbow of possible colors: yellow, red, violet, green, deep blue and blue-green cyanobacteria are known. Cyanobacteria may be single-celled or colonial. Depending upon the species and environmental conditions, colonies may form filaments, sheets or even hollow balls. Some filamentous colonies show the ability to differentiate into three different cell types. Vegetative cells are the normal, photosynthetic cells formed under favorable growing conditions. Climate-resistant spores may form when environmental conditions become harsh. A third type of cell, a thick-walled heterocyst, contains the enzyme nitrogenase, vital for nitrogen fixation. Heterocyst-forming species are able to “fix” nitrogen gas, which cannot be absorbed by plants, into ammonia (NH₃), nitrites (NO₂) or nitrates (NO₃), which can be absorbed by plants and converted to protein and nucleic acids.

The terms “complementary” or “complementarity” refer to the pairing of bases, purines and pyrimidines, that associate through hydrogen bonding in double stranded nucleic acid. The following base pairs are complementary: guanine and cytosine; adenine and thymine; and adenine and uracil. As used herein the terms include complete and partial complementarity.

The term “contacting” refers to the exposure of at least one substance or surface to another substance or surface. For example, by adding the aqueous solution to the nanopore, an aqueous solution can contact the walls of a nanopore.

The term “continuous” means that the cell's production and excretion of the product continues uninterrupted for 12, 24, 36, 48, 60, or 72 hours, or 4, 5, 6, 7, 8, 9, 10, 11, 12, or 13 days, or 2, 3, 4, 5, 6, 7, 8, 9, 10, 11, or 12 or more weeks.

The term “control level” is the standard by which a change is measured. For example, the control is not subjected to the experiment, but is instead subjected to a defined set of parameters. A control can be based on pre- or post-treatment levels.

The term “diffusivity” refers, in part, to the movement of hydrophilic waxy products within the aqueous cell cytoplasm. Because FAEEs have very low aqeuous solubility, FAEEs are expected to interact primarily with membranes. But, FAEEs were also shown to interact with phospholipid bilayers. (Bird et al., 1996). Hence, diffusivity also refers to the affinity of the cell's product, e.g., FAEEs, for the transmembrane pump as well as the resulting rate of the cell's excretion of that product via the transmembrane pump.

The term “downstream region” means a segment of a polynucleotide that is 3′ to a point of reference on the same polynucleotide.

The term “expression cassette” means a genetic module comprising a gene and the regulatory regions necessary for its expression, which may be incorporated into a vector. The expression cassette can further comprise an operably linked targeting sequence, transit or secretion peptide coding region capable of directing transport of the protein produced. The expression cassette can also further comprise a nucleotide sequence encoding a selectable marker and a purification moiety.

The term “harvesting” refers to any process by which the cell's excreted product may be collected from the cell medium. Harvesting includes but is not limited to skimming. Harvesting is non-lethal, i.e., the harvesting process does not kill the cells.

The terms “higher,” “increases,” “elevates,” or “elevation” refer to increases above basal levels, e.g., as compared to a control.

The term “homology” refers to the relationship between two or more polypeptide sequences or two or more polynucleotide sequences, as determined by comparing the sequences. In the art, “homology” also means the degree of sequence relatedness between polypeptide or polynucleotide sequences, as determined by the match between strings of such sequences. “Homology” can be readily calculated by known methods including, but not limited to, those described in Computational Molecular Biology (1988); Biocomputing. Informatics and Genome Projects (1993); Computer Analysis of Sequence Data, Part I (1994); Sequence Analysis in Molecular Biology (1987); Sequence Analysis Prime (1991); and Carillo and Lipman (1988). Methods to determine homology are designed to give the largest match between the sequences tested. Moreover, methods to determine homology are codified in publicly available programs. Computer programs which can be used to determine identity/homology between two sequences include, but are not limited to, GCG (Devereux et al., 1984; suite of five BLAST programs, three designed for nucleotide sequences queries (BLASTN, BLASTX, and TBLASTX) and two designed for protein sequence queries (BLASTP and TBLASTN) (Coulson 1994; Birren et al., 1997). The BLAST X program is publicly available from NCBI and other sources (BLAST Manual; Altschul et al., 1990). The well known Smith Waterman algorithm can also be used to determine homology.

The term “hybridization” refers to a process in which a strand of nucleic acid joins with a complementary strand through base pairing. The conditions employed in the hybridization of two non-identical, but very similar, complementary nucleic acids vary with the degree of complementarity of the two strands and the length of the strands. Thus, the term contemplates partial as well as complete hybridization. Such techniques and conditions are well known to practitioners in this field and further described herein. Methods for hybridization and washing are well known in the art and can be found in standard references in molecular biology such as those cited herein. In general, hybridizations are usually carried out in solutions of high ionic strength (6×SSC or 6×SSPE) at a temperature 20-25° C. below the Tₘ. High stringency wash conditions are often determined empirically in preliminary experiments, but usually involve a combination of salt and temperature that is approximately 12-20° C. below the Tₘ. One example of such wash conditions is 5×SSC, 50% formamide at 42° C. An example with higher stringency conditions is 1×SSC at 60° C. Another example of high stringency wash conditions is 0.1×SSPE, 0.1% SDS at 42° C. (Meinkoth and Wahl, 1984). An example of even higher stringency wash conditions is 0.1×SSPE, 0.1% SDS at 50-65° C. In one aspect, high stringency washing is carried out under conditions of 1×SSC and 60° C. It is well known to those of ordinary skill in the art that different compositions can result in equal stringency conditions for hybridization depending on well known factors such as the concentration of Na⁺, the % formamide, the temperature, the Tₘ of the hybrid to be formed, and the composition of the hybrid, e.g., DNA-DNA, DNA-RNA, or RNA-RNA. Thus the invention also encompasses nucleotide sequences that hybridize under conditions equivalent to those described above.

The terms “low,” “lower,” “reduces,” or “reduction” refer to decreases below basal levels, e.g., as compared to a control.

The term “optional” or “optionally” means that the subsequently described event or circumstance may or may not occur, and that the description includes instances in which the said event or circumstance occurs, and includes instances in which the said event or circumstances does not occur.

The terms “peptide” and “protein” are used interchangeably and mean a compound that consists of two or more amino acids that are linked by means of peptide bonds.

The terms “polynucleotide” and “oligonucleotide” are used interchangeably and mean a polymer of at least 2 nucleotides joined together by phosphodiester bonds and may consist of either ribonucleotides or deoxyribonucleotides.

The term “product” refers to the waxy product excreted into the extracellular matrix by the cell. Products include, but are not limited to, antibiotics, amphipathic pharmaceuticals, hydrophobic dyes, hydrophobic pigments, hydrophobic fragrances, hydrophobic flavors, monomers and oligomers for polymer assembly, vegetable oils, hydrophobic vitamins and vitamin precursors, hormones, and other biologically active molecules.

The term “protein” includes the forms of the protein to which one or more substituent groups have been added. A substituent is an atom or group of atoms that is introduced into a molecule by replacement of another atom or group of atoms. Such groups include, but are not limited to, lipids, phosphate groups, sugars and carbohydrates. Thus, the term protein includes, for example, lipoproteins, glycoproteins, phosphoproteins and phospholipoproteins.

The term “proteogenic” indicates that the amino acid can be incorporated into a peptide, polypeptide, or protein in a cell through a metabolic pathway.

The term “recombinant nucleic acid” is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. Thus, for example, products made by transforming cells with any unnaturally occurring vector is encompassed, as are nucleic acids comprising sequences derived using any synthetic oligonucleotide process. Such is often done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design.

The term “recombinant protein” means that the protein, whether comprising a native or mutant primary amino acid sequence, is obtained by expression of a gene carried by a recombinant DNA molecule in a cell other than the cell in which that gene and/or protein is naturally found. In other words, the gene is heterologous to the host in which it is expressed. It should be noted that any alteration of a gene, including the addition of a polynucleotide encoding an affinity purification moiety to the gene, makes that gene unnatural for the purposes of this definition, and thus that gene cannot be “naturally” found in any cell.

The terms “secretion sequence” or “signal peptide” or “signal sequence” means a sequence that directs newly synthesized secretory or membrane proteins to and through membranes of the endoplasmic reticulum, or from the cytoplasm to the periplasm across the inner membrane of bacteria, or from the matrix of mitochondria into the inner space, or from the stroma of chloroplasts into the thylakoid. Fusion of such a sequence to a gene that is to be expressed in a heterologous host ensures secretion of the recombinant protein from the host cell.

The term “sequence” means the linear order in which monomers occur in a polymer, for example, the order of amino acids in a polypeptide or the order of nucleotides in a polynucleotide.

The term “transgene” refers to any piece of DNA which is inserted by artifice into a cell, and becomes part of the genome of the organism (i.e., either stably integrated or as a stable extra-chromosomal element) which develops from that cell. Such a transgene can include a gene which is partly or entirely heterologous (i.e., foreign) to the transgenic organism, or may represent a gene homologous to an endogenous gene of the organism. Included within this definition is a transgene created by the providing of an RNA sequence that is transcribed into DNA and then incorporated into the genome. The transgenes disclosed herein can include DNA sequences that encode the fluorescent or bioluminescent protein that may be expressed in a transgenic non-human animal.

The term “transgenic” is used to describe an organism that includes exogenous genetic material within all of its cells. The term includes any organism whose genome has been altered by in vitro manipulation of the early embryo or fertilized egg or by any transgenic technology to induce a specific gene knockout.

The term “upstream region” means a segment of a polynucleotide that is 5′ to a point of reference on the same polynucleotide.

### 2. COMPOSITIONS

A. Nucleic Acids

There are a variety of molecules disclosed herein that are nucleic acid based, including for example the nucleic acids that encode AEAT, PDC, and ADH, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

In one aspect, the present invention involves recombinant polynucloetides comprising the isolated sequence of AEAT along with other sequences. Such recombinant polynucleotides are the invention provides an isolated polynucleotide encoding a polypeptide having acyl-CoA: ethanol acyltransfers (AEAT) activity, and having a nucleotide sequence at least about 60% homologous to SEQ ID NO. 1 or 2, for example. It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

As this specification discusses various proteins and protein sequences, it is understood that the nucleic acids that can encode those protein sequences are also disclosed. This would include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence is set forth in SEQ ID NO. 1. For example, another nucleic acid sequence that encodes the same protein sequence is set forth in SEQ ID NO. 2. It is also understood that while no amino acid sequence indicates what particular DNA sequence encodes that protein within an organism, where particular variants of a disclosed protein are disclosed herein, the known nucleic acid sequence that encodes that protein in the particular xxx from which that protein arises is also known and herein disclosed and described.

Also disclosed are isolated peptides comprising an amino acid segment comprising, for example, the amino acid sequence of SEQ ID NO. 3 or SEQ ID NO. 4, or the amino acid sequence of SEQ ID NO. 3 or SEQ ID NO. 4 that have one or more conservative amino acid substitutions. For example, the peptide can have 1, 2, 3, 4, or 5 conservative amino acid substitutions. One of skill in the art is readily able to assess which amino acids can be substituted and retain the function of the peptide.

i. Wax Ester Synthase/Acyl-Coenzyme A: Diacylglycerol Acyltransferase (WS/DGAT)

Intracytoplasmic storage lipid accumulation in Acinetobacter baylyi is mediated wax ester synthase/acyl-coenzyme A: diacylglycerol acyltransferase. (Vaneechoutte et al., 2006). wax ester synthase/acyl-coenzyme A: diacylglycerol acyltransferase (WS/DGAT) is the product of the atfA gene. WS/DGAT exhibits an extremely low acyl acceptor molecule specificity in vitro. The remarkably broad substrate range of WS/DGAT includes (1) short chain-length up to very long chain length linear primary alkyl alcohols, (2) cyclic, phenolic and secondary alkyl alcohols, (3) diols and dithiols, and (4) mono- and diacylglycerols as well as sterols. (Kalscheuer et al., 2003, 2004). A representative amino acid sequence for WS/DGAT in Acinetobacter sp. strain ADP1 is presented in SEQ ID NO. 7.

ii. Acyl-CoA: Ethanol Acyltransferase

The synthesis of FAEE from ethanol and fatty-acyl CoA molecules is an important element of the FAEE synthesis and efflux pathway. Previously, Nakao et al. identified an enzyme having acyl-CoA: ethanol acyltransferase (AEAT) activity in Saccharomyces cerevisiae. (WO 2007/097091). The present invention identifies two nucleic acid sequences in the human genome that demonstrates the AEAT activity. These nucleic acid sequences comprise the nucleotide sequences listed in SEQ ID. NO. 1 and SEQ ID NO. 2.

iii. Alcohol Dehydrogenase

Alcohol dehydrogenase (ADH), or aldehyde reductase, is an enzyme found in a variety of species ranging from Escherichia coli to Ursus arctos (Brown Bear) to Saccharomyces cerevisiae to human. ADH is a member of a general class of enzymes called oxidoreductases, a class of enzymes that utilize the same basic mechanism to form aldehydes or ketones from an alcohol. ADH also catalyzes the oxidation of many different alcohols including: primary, secondary, cyclic secondary, or hemi-acetal. Biologically, in humans, ADH is active as a dimer. Each subunit has two domains, a NAD binding domain, and an alcohol (substrate) binding domain. In the obligately fermentative bacterium Zymomonas mobilis, the regeneration of NAD* depends upon two enzymes, i.e., pyruvate decarboxylase and alcohol dehydrogenase. Z. mobilis is the only known obligately fermentative procaryote which utilizes an Entner-Doudoroff pathway for glycolysis and, like Saccharomyces cerevisiae, produces ethanol and carbon dioxide as dominant fermentation products. In Z. mobilis, ADH is a tetramer with a monomeric molecular weight 45 between 31,000 and 38,000 (Hoppner and Doelle, 1983; Kinoshita et al., 1985; Neale et al., 1986; Scopes 1983; Wills et al., 1981). This isozyme is quite unusual, containing iron rather than zinc in its active site (Neale et al., 1986; Scopes 1983). Furthermore, in Z. mobilis, ADH has a high degree of specificity for ethanol as a substrate (Kinoshita et al., 1985; Neale et al., 1986; Wills et al., 1981). A representative amino acid sequence for ADH B in Z. mobilis is presented in SEQ ID NO. 5.

iv. Pyruvate Decarboxylase

Pyruvate decarboxylase (PDC) is a homotetrameric enzyme that catalyses the decarboxylation of pyruvic acid to acetaldehyde and carbon dioxide. It is also called 2-oxo-acid carboxylase, alpha-ketoacid carboxylase, and pyruvic decarboxylase. In aerobic conditions, this enzyme is the first in a three enzyme complex known as the pyruvate dehydrogenase complex, which converts pyruvate (the product of glycolysis) into acetyl CoA. In anaerobic conditions, this enzyme is part of the fermentation process that occurs in yeast to produce alcohol (this enzyme is not in animals). Pyruvate decarboxylase starts this process by converting pyruvate into acetaldehyde and carbon dioxide. To do this, two thiamine pyrophosphate (TPP) and two Magnesium ions are required as cofactors. One key feature about this enzyme is its structure. The protein has a beta-alpha-beta structure, yielding parallel beta-sheets. These domains together provide a shape and structure for the thiamine pyrophosphate to bind to it. The dimer of pyruvate decarboxylase is stabilized by the two TPP and two Mg²⁺ molecules that hold them together. The two active sites of the protein consist of the thiamine pyrophosphate group and 20 amino acids. Although the amino acids are not highly conserved for the entire protein from species to species, the amino acids at the active site are highly conserved. The two glutamic acids (477 and 51) are vital to the reaction mechanism. Glu 477 stabilizes the thiazolium ring of TPP when it binds with pyruvate. Likewise, the Glu 51 is essential for cofactor binding and catalytic activity. Studies have shown that a mutation at this residue yields little to no catalytic activity. In anaerobic conditions, the enzyme has a twofold task: first to convert pyruvate into hydroxyethyl-TPP and next to transfer the hydroxyethyl group attached to TPP to the lipoamide from the E2 component of the pyruvate dehydrogenase complex. This transfer ultimately brings pyruvate decarboxylase back to its native form, ready to catalyze the next reaction. This reaction takes place in the mitochondrial matrix. In yeast, pyruvate decarboxylase acts independently during fermentation and releases the 2-carbon fragment as acetaldehyde plus carbon dioxide. Pyruvate decarboxylase creates the means of CO₂ elimination, which the cell dispels. (Hoberg 2007). The enzyme is also means to create ethanol, which is used as an antibiotic to eliminate competing organisms. (Dyda et al., 1993). The enzyme is necessary to help the decarboxylation of alpha-keto acids because there is a build-up of negative charge that occurs on the carbonyl carbon atom in the transition state; therefore, the enzyme provides the suitable environment for TPP and the alpha-keto acid (pyruvate) to meet. (Dyda et al., 1993). A representative amino acid sequence for PDC in Z. mobilis is presented in SEQ ID NO. 6.

v. Resistance-Nodulation-Division Transporter (RND)

Among the five known families of multidrug transporters, the resistance-nodulation-division (RND) family tends to play major roles in the intrinsic resistance of gram-negative bacteria. For example, some RNDs can extrude a very large range of compounds. The Escherichia coli genome contains several genes coding for RND transporters. One example is AcrB, which provides the major mechanism for multiple drug resistance in E. coli. AcrB forms a multiprotein complex with AcrA, a periplasmic protein of the membrane fusion protein family and the complex is likely to include an outer membrane channel protein, TolC. TolC, an outer-membrane beta-barrel protein, is multi-functional and has known homologs in Synechocystis. This tripartite construction allows the transporter to pump out drugs directly into the medium, rather than into the periplasm, making the complex much more efficient in producing resistance. Another example in E. coli is AcrF. In Pseudomonas aeruginosa, at least MexB, MexD, and MexF have been identified as RND transporters, and MexAB-OprM has been identified as a homolog to the AcrAB-TolC complex. Similarly, at least 12 efflux pumps of the RND family have been identified in Pseudomonas putida, including TtgB, TtgE, TtgH, SrpB, and ArpB. Tripartite RND transporters require an inner membrane efflux transporter, a periplasmic membrane fusion protein, and an outer membrane protein channel for function.

B. Transgenic Inserts

Transgenic cells can be prepared using any convenient protocol that provides for stable integration of the transgene into the bacterial genome in a manner sufficient to provide for the requisite spatial and temporal expression of the transgene. It is understood that the various genes disclosed herein can be used in any order, or in any combination, in a contract. A number of different strategies can be employed to obtain the integration of the transgene with the requisite expression pattern. Generally, methods of producing the subject transgenic cyanobacteria involve stable integration of the transgene into the cyanobacterial genome. Stable integration is achieved by first introducing the transgene into a cyanobateria. The transgene is generally present on a suitable vector, such as a plasmid. Transgene introduction may be accomplished using any convenient protocol, where suitable protocols include: electroporation, microinjection, vesicle delivery, e.g. liposome delivery vehicles, and the like. Following introduction of the transgene into the cell(s), the transgene is stably integrated into the genome of the cell. Stable integration may be either site specific or random, but is generally random.

Where integration is random, the transgene is typically integrated with the use of transposase. In such aspects of the invention, the transgene is introduced into the cell(s) within a vector that includes the requisite P element, terminal 31 base pair inverted repeats. Where the cell into which the transgene is to be integrated does not comprise an endogenous transposase, a vector encoding a transposase is also introduced into the cell, e.g., a helper plasmid comprising a transposase gene, such as pTURBO. (Steller & Pirrotta, 1986). Methods of random integration of transgenes into the genome of a target Drosophila melanogaster cell(s) are disclosed in U.S. Pat. No. 4,670,388, the disclosure of which is herein incorporated by reference.

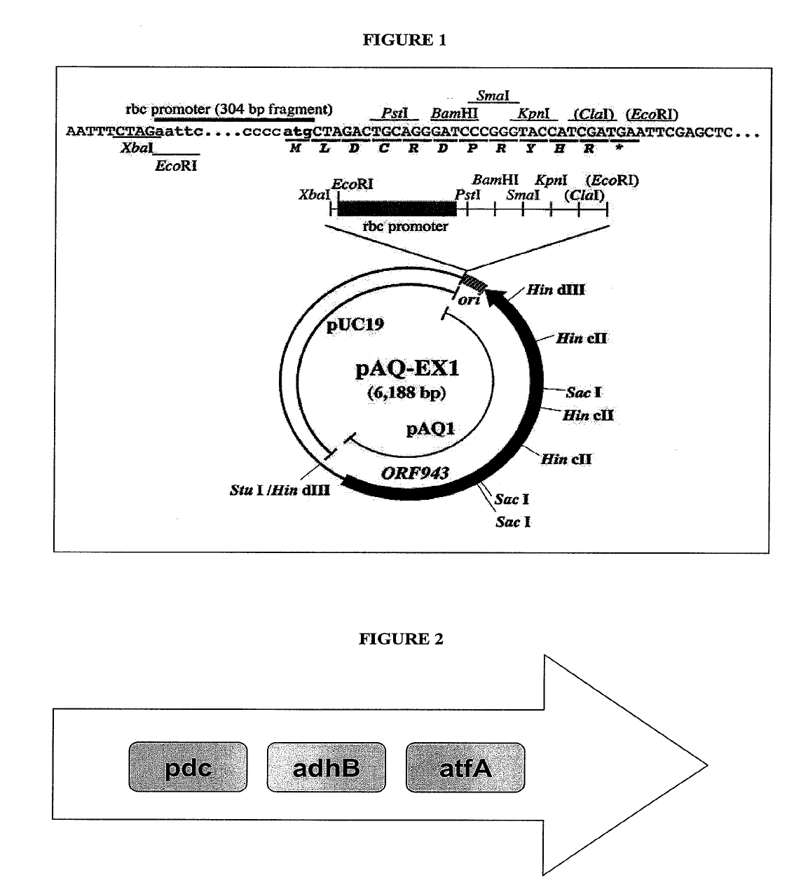

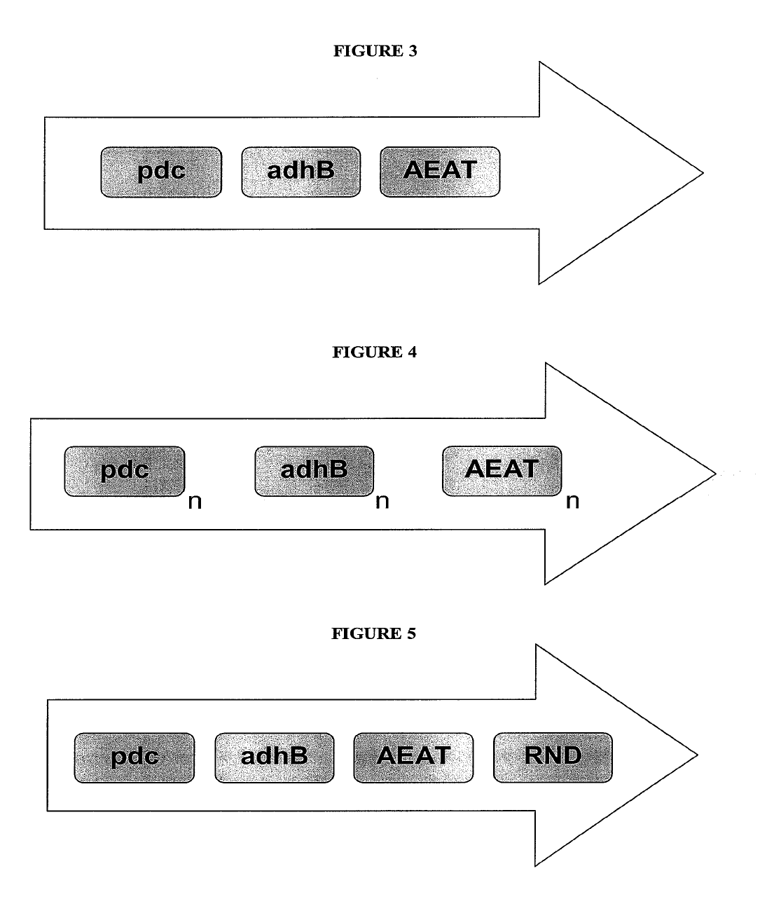

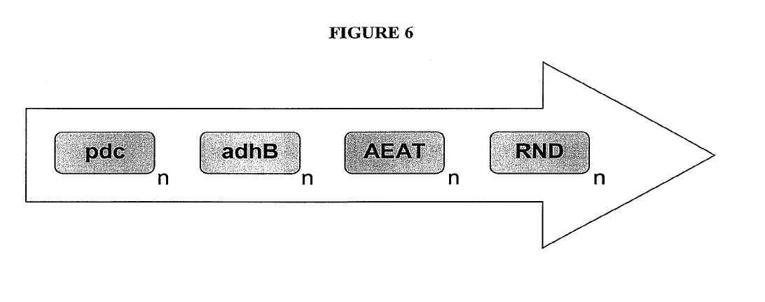

In some aspects, the present invention utilizes the plasmid pAQ-EX1. (See FIG. 1). The complete amino acid sequence for this expression vector is listed in SEQ ID NO. 8, and the complete nucleotide sequence for this expression vector is listed in SEQ ID NO. 9. The plasmid pAQ-Ex1 carries four unique restriction sites in MCS: BamHI, SmaI, pstl, and Kpnl. For example, in some aspects of the invention, three genes are inserted downstream of the rbcL promoter (the large subunit of ribulose-1,5-bisphosphate carboxylase/oxygenase) located in MCS. First, pdc amplified from genomic DNA 40 bp upstream and 29 bp downstream of pdc gene, and the PCR product contains pstl and pvuI sites. Second, adhB amplified from genomic DNA 43 bp upstream and 27 bp downstream of adhB gene, and the PCR product contains pvuI and BamHI sites. Third, atfA amplified from genomic DNA 21 bp upstream and 130 bp downstream of atfA gene, and the PCR product contains BamHI and XmaI sites. (FIG. 2).

In some aspects, the present invention uses the following transformation protocol. First, the cell were cultured in 100 mL of BG-11 liquid medium at 28° C. under cool white fluorescent light and subcultured at mid-exponential phase of growth. To 1.0 mL of cell suspension containing 2×10⁸ cells (for Synechoccus sp. PCC 7942, A₇₃₀=0.5) of 10⁹ cells (for Synechoccus sp. PCC 6301, A₇₃₀=2.5), which were cultured at the mid-exponential phase of growth, 0.5 or 1.0 μg of donor DNA (in 10 mM Tris/1 mM EDTA, pH8.0) was added, and the mixture was incubated in the dark at 26° C. overnight. After incubation for further 6 hours in the light, the transformants were directly selected on BG-11 agar plates containing 1.5% agar, 1 mM sodium thiosulfate and 0.5 μg/mL Ap. The transformants frequency was calculated by counting the number of transformants after 15 days.

In those aspects in which the transgene is stably integrated in a random fashion into the cell's genome, means are also provided for selectively expressing the transgene at the appropriate time during development of the cell. In other words, means are provided for obtaining targeted expression of the transgene. To obtain the desired targeted expression of the randomly integrated transgene, integration of particular promoter upstream of the transgene, as a single unit in the P element vector may be employed. Alternatively, a transactivator that mediates expression of the transgene may be employed. Of particular interest is the GAL4 system as described herein.

As mentioned above, an example of a transposable element is the P element. (Rubin and Spradling, 1982; Spradling and Rubin, 1982). The gene of interest is placed between P element ends, usually within a plasmid, and injected into pre-blastoderm embryos in the presence of transposase. This P element, with the gene as cargo, then transposes from the plasmid to a random chromosomal site. P-elements are small transposons with terminal 31-bp inverted repeats, and the element generates 8-bp direct repeats of target DNA sequences upon insertion. The complete element is 2907 bp and is autonomous because it encodes a functional transposase. Incomplete P elements have lost the transposition ability because the transposase has been mutated. But if a complete (autonomous) element exists in the same cell as an incomplete (non-autonomous) element, then the incomplete element can transpose because of the presence of the transposase in the cell. (Ashburner 1989, Spradling 1986).

Examples of other transposable elements include piggyBAC and Mariner. The piggyBAC element is 2.4 kb in length and terminates in 13 bp perfect inverted repeats, with additional internal 19 bp inverted repeats located asymmetrically with respect to the ends (Cary et al., 1989). The initial sequence analysis of the piggyBAC element revealed a potential RNA polymerase II promoter sequence configuration, typical Kozak translational start signal, and two apparently overlapping long open reading frames. Mariner belongs to a superfamily of DNA-based transposons that includes the C. elegans Tcl element. It is small (1.3 kb), encoding one protein (the transposase) flanked by 28 bp inverted repeats. (Medhora et al., 1991)

Another suitable promoter of the Drosophila origin includes the Drosophila metallothionein promoter (Lastowski-Perry et al., 1985). This inducible promoter directs high-level transcription of the gene in the presence of metals, e.g., CuSO₄. This is in direct contrast to the use of the mammalian metallothionein promoter in mammalian cells in which the regulatory effect of the metal is diminished as copy number increases. In the Drosophila expression system, this retained inducibility effect increases expression of the gene product in the Drosophila cell at high copy number. Examples of expression systems useful for this method include, but are not limited to, the gene switch protocol and the Haig Keshishian RU486 method. The gene switch system, also known as the Ga180 system, works as follows: a temperature sensitive allele of Ga180 is combined with the usual gal4-UAS constructs: a gal4 driver, a tissue-specific reporter and a Ga180 cassette to repress Gal4 and stop its ectopic gene expression. It takes approximately 3-6 hours to get peak expression and then 15 hours to turn peak expression off, so if toxicity were a problem with any of these constructs, this can be a useful misexpression system. The Haig Keshishian system uses RU486, which is also referred to as a gene switch, and is similar to a Ga14 system developed using estrogen receptors, allowing steroid hormones to act as the activating switch. This system can be thought of as a modified Gal4 system, dependent on the presence of mifepristone (RU486). Transgenic lines express this modified Gal4 protein, which remains inactive until bound to mifepristone; it can then bind to UAS sequences as would a normal Gal4 molecule and initiate transcription. (Osterwalder et al., 2001).

C. Protein Variants

The present invention also relates to proteins encoded by the isolated polynucleotides. As used herein the term protein includes fragments, analogs and derivatives of the wax ester synthase-like protein. The terms “fragment,” “derivative” and “analog” as used herein mean a polypeptide that retains essentially the same biological function or activity as the acyl-CoA:ethanol acyltransferase (AEAT) encoded by the sequence of the present invention. For example, an analog includes a proprotein which can be cleaved to produce an active mature protein. The protein of the present invention can be a natural protein, a recombinant protein or a synthetic protein or a polypeptide.

The fragment, derivative, or analog of the proteins encoded by the polynucleotide sequence of the present invention may be, for example and without limitation, (i) one in which one or more amino acid residues are substituted with a conserved or non-conserved amino acid residue, and such substituted amino acid residue may or may not be one encoded by the genetic code; (ii) one in which one or more of the amino acid residues includes a substituent group; (iii) one in which the mature protein is fused to another compound such as a compound to increase the half-life of the protein; (iv) one in which additional amino acids are fused to the protein to aid in purification or in detection and identification; or (v) one in which additional amino acid residues are fused to the protein to aid in modifying tissue distribution or localization of the protein to certain locations such as the cell membrane or extracellular compartments.

Those of ordinary skill in the art are aware that modifications in the amino acid sequence of a peptide, polypeptide, or protein can result in equivalent, or possibly improved, second generation peptides, etc., that display equivalent or superior functional characteristics when compared—to the original amino acid sequence. The present invention accordingly encompasses such modified amino acid sequences. Alterations can include amino acid insertions, deletions, substitutions, truncations, fusions, shuffling of subunit sequences, and the like, provided that the peptide sequences produced by such modifications have substantially the same functional properties as the naturally occurring counterpart sequences disclosed herein. Biological activity or function can be determined by, for example, the ability of the protein to increase wax ester production in a host cell as depicted in the examples below.

Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 2
Amino Acid
Original Residue Exemplary Conservative
Substitutions (others are known in the art)

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

TABLE 1
Amino Acid Abbreviations
Substitutions

| Amino Acid | Abbreviations |
|---|---|
| alanine | Ala or A |
| allosoleucine | A or Ile |
| arginine | Arg or R |
| asparagine | Asn or N |
| aspartic acid | AspD |
| cysteine | Cys or C |
| glutamic acid | Glu or E |
| glutamine | Gln or K |
| glycine | Gly or G |
| histidine | His or H |
| isolelucine | Ile or I |
| leucine | Leu or L |
| lysine | Lys or K |
| phenylalanine | Phe or F |
| proline | Pro or P |
| pyroglutamic acid | Glu |
| serine | Ser or S |
| threonine | Thr or T |
| tyrosine | Tyr or Y |
| tryptophan | Trp or W |
| valine | Val or V |

### 3. METHODS

A. Delivery of the Compositions to Cells

The present invention includes recombinant constructs comprising the isolated polynucleotide sequence of the present invention. The constructs can include a vector, such as a plasmid or viral vector, into which the sequence of the present invention has been inserted, either in the forward or reverse orientation. The recombinant construct further comprises regulatory sequences, including for example, a promoter operatively linked to the sequence. Large numbers of suitable vectors and promoters are known to those skilled in the art and are commercially available. It will be understood by those skilled in the art, however, that other plasmids or vectors may be used as long as they are replicable and viable or capable of expressing the encoded protein in the host.

Generally, these methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff et al., 1990 and Wolff 1991.

i. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus. (Ram et al., 1993).

As used herein, plasmid or viral vectors are agents that transport the disclosed nucleic acids, such as AEAT into the cell without degradation and include a promoter yielding expression of the gene in the cells into which it is delivered. In some aspects, the vectors are derived from either a virus or a retrovirus. For example, viral vectors include but are not limited to Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred aspect is a viral vector which has been engineered so as to suppress the immune response of the host organism, elicited by the viral antigens.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a. Retroviral Vectors

A retrovirus is an animal virus belonging to the virus family of Retroviridae, including any types, subfamilies, genus, or tropisms. Retroviral vectors, in general, are described by Verma 1985, which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan 1993, which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b. Adenoviral Vectors

The construction of replication-defective adenoviruses has been described (Berkner et al., 1987; Massie et al., 1986; Haj-Ahmad et al., 1986; Davidson et al., 1987; Zhang et al., 1993). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy 1993; Kirshenbaum 1993; Roessler 1993; Moullier 1993; La Salle 1993, Gomez-Foix 1992; Rich 1993; Zabner 1994; Guzman 1993; Bout 1994; Zabner 1993; Caillaud 1993; and Ragot 1993). Recombinant adenoviruses achieve gene transduction by binding to specific cell surface receptors, after which the virus is internalized by receptor-mediated endocytosis, in the same manner as wild type or replication-defective adenovirus. (Chardonnet and Dales, 1970; Brown and Burlingham, 1973; Svensson and Persson, 1985; Seth et al., 1984; Seth et al., 1984; Varga et al., 1991; Wickham et al., 1993).

A viral vector can be one based on an adenovirus which has had the E1 gene removed and these virons are generated in a cell line such as the human 293 cell line. In another preferred aspects, both the E1 and E3 genes are removed from the adenovirus genome.

c. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred aspect of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector. The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

d. Large Payload Viral Vectors

Molecular genetic experiments with large human herpesviruses have provided a means whereby large heterologous DNA fragments can be cloned, propagated and established in cells permissive for infection with herpesviruses (Sun et al., 1994; Cotter and Robertson, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

ii. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC—cholesterol) or anionic liposomes are disclosed. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al., 1989; Feigner et al., 1987; U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter et al., 1991; Bagshawe 1989; Bagshawe et al., 1988; Senter et al., 1993; Battelli et al., 1992; Pietersz and McKenzie, 1992; and Roffler et al., 1991). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., 1989; Litzinger and Huang, 1992). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed. (Brown and Greene, 1991).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

iii. In Vivo/Ex Vivo

As described above, the compositions can be administered in a pharmaceutically acceptable carrier and can be delivered to the cells in vivo and/or ex vivo by a variety of mechanisms well known in the art (e.g., uptake of naked DNA, liposome fusion, intramuscular injection of DNA via a gene gun, endocytosis, and the like). If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

iv. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a. Viral Promoters and Enhancers Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication. (Fiers et al., 1978). The immediate early promoter of the human cytomegalovirus is conveniently obtained as a HindIII E restriction fragment (Greenway et al., 1982). Of course, promoters from the host cell or related species also are useful herein.

Enhancer generally refers to a sequence of DNA that functions at no fixed distance from the transcription start site and can be either 5′ (Laimins et al., 1981) or 3′ (Lasky et al., 1983) to the transcription unit. Furthermore, enhancers can be within an intron (Banerji et al., 1983) as well as within the coding sequence itself (Osborne et al., 1984). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences from mammalian genes are now known (globin, elastase, albumin, fetoprotein and insulin), one typically uses an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs

In some aspects, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. In some aspects, it is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

In some aspects of the present invention, the promoters are provided by the bacterial cell itself. Furthermore, in some aspects, the transgenic insert contains ribosome binding sites from other cells to increase expression, which cells include but are not limited to the cyanobacteria Synechoccus sp. PCC 7492, for example.

b. Markers

In some aspects of the present invention, the marker can confer antibiotic resistance to the cell. The plasmid, for example, can contain antibiotic resistance genes, including but not limited to the Ampicillin resistance gene (AmpR).

The viral vectors can include nucleic acid sequence encoding a marker product. This marker product is used to determine if the gene has been delivered to the cell and once delivered is being expressed. Examples of markers include, but are not limited to, alkaline phosphatase (AP), myc, hemagglutinin (HA), B glucuronidase (GUS), luciferase, and green fluorescent protein (GFP). In some aspects, the vectors contain structural genes providing resistance to kanamycin and nalidixic acid. In addition, expression vectors can also contain marker sequences operatively linked to a nucleotide sequence for a protein that encodes an additional protein used as a marker. The result is a hybrid or fusion protein comprising two linked and different proteins. The marker protein can provide, for example, an immunological or enzymatic marker for the recombinant protein produced by the expression vector

In some aspects, the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR cells and mouse LTK cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid (Mulligan and Berg, 1980) or hygromycin (Sugden et al., 1985). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

B. Production of Ethyl Alcohol and FAEEs/FATBEs and Other Products

The methods disclosed herein related to transformed host cells containing the constructs comprising the polynucleotide sequence of the present invention. For example, the disclosed methods relate to genetically modified photosynthetic cyanobacteria that are capable of producing ethanol. The cyanobacteria are genetically modified by the insertion of DNA fragments comprising, at the minimum, the amino acid sequences for the AEAT, PDC, and ADH. AEAT joins ethanol and C₁₆-C₁₈ fatty acyl-CoA to form long chain FAEE. Pyruvate decarboxylase converts pyruvate to acetaldehyde, and alcohol dehydrogenase converts acetaldehyde to ethanol. The in vivo production of alcohol occurs in such sufficient quantities that the alcohol becomes a reactant in the transesterification of fatty acids. The tranesterification of fatty acids produces fatty acid alcohol esters (FAEEs) or fatty acid t-butyl esters (FATBEs). FAEEs and FATBEs are high value waxy products. Hence, the methods disclosed herein comprise producing high value fatty acid ethyl esters (FAEEs) in a genetically modified cell, wherein the cell comprises at least one transgenic insert comprising acyl-CoA: ethanol acyltransferase (AEAT).

The methods disclosed herein also comprise producing other products in a genetically modified cell, wherein the cell comprises at least one transgenic insert comprising the polynucleotide sequent of interest.

The methods disclosed herein also provide for the production by the cell of large quantities of product, wherein the modified cells carry amplified copies of the sequence of interest. In this method, the sequence is contained in a vector that carries a selectable marker and transfected into the host cell or the selectable marker is co-transfected into the host cell along with the sequence of interest. Lines of host cells are then selected in which the number of copies of the sequence have been amplified. A number of suitable selectable markers will be readily apparent to those skilled in the art. For example, the dihydrofolate reductase (DHFR) marker is widely used for co-amplification. Exerting selection pressure on host cells by increasing concentrations of methotrexate can result in cells that carry up to 1000 copies of the DHFR gene.

C. Excretion of FAEEs/FATBEs into the Extracellular Matrix

The methods disclosed herein also assist lipid droplets to locate close to transmembrane pumps. Hence, the disclosed methods reduce the barrier to diffusion through the cell's aqueous cytoplasm, and increase the cell's ability to excrete the products. The cell continuously produces valuable product for excretion. The cell's excretion of the product allows the product to agglomerate in the extracellular matrix.

Furthermore, the disclosed methods comprise harvesting the product from the extracellular matrix without killing the cell. The disclosed methods comprise a harvesting process that reduces the byproduct yield of non-waxy cell debris, which requires expensive waste treatment facilities. Thus, the disclosed methods eliminate the need for extraction with solvent methods, and greatly reduce the cost of separate the product from other components of the culture. Unlike a traditional industrial application, in the use of the disclosed methods, in the present invention, there is no need to procure, transport, or store hazardous ethanol. Similarly, the disclosed methods avoid the need for traditional industrial transesterification, which requires toxic and expensive catalysts. Hence, the disclosed methods convert the semi-batch process, i.e., grow and then kill cells to harvest product, to a continuous process. Such a continuous process is more readily scaled to an industrial size application.

The methods disclosed herein also contemplate the overexpression of an RND pump, wherein that RND achieves a continuous efflux of the cell's products. While solubility and toxicity concerns govern the circumstances around an individual product, the disclosed RND pump is adaptable to a synthesis pathway other than that of FAEE pathway.

In some aspects, the methods disclosed herein further comprise the AcrB gene. Here, the AcrB gene is modified by the addition of an appropriate cell signal sequence such that the protein product is targeted to the cell plasma membrane and functionally docks with TolC and AcrA in the periplasmic space. The modified AcrB gene is then inserted into a cell. The AcrB gene product acts in conjunction with the TolC and AcrA proteins to form a membrane-spanning complex that excretes FAEEs and other products.

In some aspects, the methods disclosed herein further comprise the acetyl-CoA carboxylase genes which include but are not limited to accA, accB, accC and accD. These genes are inserted into the cell such that these genes can be expressed in combination with other inserted genes. These genes produced a superabundance of intracellular malonyl-CoA through the metabolism of photosynthetically produced acetyl-CoA. The increased synthesis of malonyl-CoA enhances the ability of the cell to replace acyl-CoA consumed in the biosynthesis of FAEEs and other products.

i. Transmembrane Pump

In cells, the overproduction of efflux transporters leads to a depressed levels of antibiotics or chemotherapeutics. Hence, the overproduction of efflux transporters contributes to drug resistance during infection or cancer treatment. (Li and Nikaido, 2004; Ambudkar et al., 2006). One type of drug efflux transporters is the ABC type. This type of drug efflux transporter uses the free energy of adenosine triphosphate (ATP) hydrolysis. ABC-type transporters are usually found in eukaryotes. Another type of transporter is the secondary transporter, which uses the proton motive force to energize the extrusion of drugs.

Among the five known families of multidrug transporters, the resistance-nodulation-division (RND) family tends to play major roles in the intrinsic resistance of gram-negative bacteria. For example, some RNDs can extrude a very large range of compounds. The Escherichia coli genome contains several genes coding for RND transporters. One example is AcrB, which provides the major mechanism for multiple drug resistance in E. coli. AcrB forms a multiprotein complex with AcrA, a periplasmic protein of the membrane fusion protein family and the complex is likely to include an outer membrane channel protein, TolC. This tripartite construction allows the transporter to pump out drugs directly into the medium, rather than into the periplasm, making the complex much more efficient in producing resistance. Another example in E. coli is AcrF. In Pseudomonas aeruginosa, at least MexB, MexD, and MexF have been identified as RND transporters, and MexAB-OprM has been identified as a homolog to the AcrAB-TolC complex. Similarly, at least 12 efflux pumps of the RND family have been identified in Pseudomonas putida, including TtgB, TtgE, TtgH, SrpB, and ArpB. Tripartitte RND transporters require an inner membrane efflux transporter, a periplasmic membrane fusion protein, and an outer membrane protein channel for function.

In prokaryotes, the main drug-efflux system involves Hb/drug exchangers. In Gram-positive bacteria, the members of the major facilitator superfamily often confer drug resistance. (Markham and Neyfakh, 2001). But, the situation in Gram-negative bacteria differs. Gram-negative bacteria have resistance nodulation-cell division (RND) type efflux pumps. (Li and Nikaido, 2004; Poole 2004).

The RND pump AcrB was the first RND pump to have its structure determined. The structure of the AcrB was resolved at 3.5 Å resolution from crystals grown in a trigonal space group assigned as R32. (Pos and Diederichs, 2002; Murakami et al., 2002; Yu et al., 2003; Pos et al., 2004). 12 transmembrane α helices (TM1 to TM12) comprise the AcrB monomer. Two α-helices—TM4 and TM10—are surrounded by the other transmembrane helices of the monomer. (Murakami et al., 2002; Guan and Nakae, 2001). These monomers harbor the residues D407, D408 (TM4), and K940 (TM10), which appear to play an essential role in proton translocation.

The periplasmic part of AcrB consists of two domains: (1) the TolC docking domain (DN and DC subdomains) which is located farthest from the membrane plane, and (2) the pore domain, composed of subdomains PN1, PN2, PC1, and PC2. The TolC docking domain has a funnel-like structure narrowing to a central pore located in the pore domain. (Tamura et al., 2005). The central pore structure consists of three α helices, also referred to as pore helices. Regarding the mechanism of transport through this pump, the leading proposal envisions the diffusion of substrates via the transmembrane domains and vestibules into the central cavity and the opening of the central pore to allow the transport of the substrates through AcrB toward TolC (Koronakis et al., 2000) and export to the external medium. (Murakami et al., 2002; Murakami et al., 2004). Recently generated research implied an alternating access mechanism for transport by AcrB. (Seeger et al., 2006). This study suggested that the AcrB utilizes three different monomer conformations—open, loose, and tight—representing consecutive states in a transport cycle. (Seeger et al., 2006).

### EXAMPLES

### Design of Primers for Amplification of atfA Gene from Acinetobacter

In an experiment, the forward primer (atfAfor) for amplification of atfA was GATCCCGGGCAATCCACGCTATGCGCCCATTACATC (SEQ ID NO: 10) and the reverse primer (atfArev) for atfA was GTCGGATCCAGATCACGACTGCAATGGTTCATC (SEQ ID NO: 11).

Preparation of Medium LK and Growth of E. coli KNabc

The medium LK comprises 6 g of KCl, 10 g of Tryptone, 5 g yeast extract, and a pH of 7.5

### Growth of KNabc

In an experiment, cells from microtubes stored at −80° C. were thawed gradually on ice. A small drop of solution was inoculated into liquid medium. Cultures were grown in an incubator at 35° C. overnight.

Preparation of Competent Cells of E. coli KNabc

In an experiment, the cells were centrifuged at 10,000 rpm for 5 minutes at 4° C. The samples were then kept on ice for 10-15 min. The cells were centrifuged at 8 krpm at 4° C. for 5 min. PPT was then suspended in 16 mL of ice cold solution #1, kept on ice for 15 min, and centrifuged at 8 krpm at 4° C. for 5 min. PPT was then resuspended in 1.6 mL of solution #2, kept on ice for 15 min, divided into 200 μL aliquots, and then placed at −80° C.

Transformation of E. coli KNabc with pAQ-EX1 Plasmid

In an experiment, cells were thawed on ice for 15 min and then 10 μL of 10 ng/μL pAQ-EX1 plasmid was added. The cells were placed on ice for 30 min. The cells were heat shocked at 42° C. for 90 sec and then kept on ice for 2-3 minutes. 500 μL of LK medium was added and the cells were incubated in a water bath at 37° C. for one hr. Cells were then plated on plates containing 60 μg/mL ampicillin. Either 10 μL, 40 μL, or 100 μL of transformant solution was added to the plate.

### Selection of Transformants and Growth of Transformants in Liquid LK Medium

In an experiment, LK medium containing 60 μg/mL amipicillin was used to select transformants. After being spread on plates, colonies (more than 400 with 40 μL of transformation solution spread on each plate) were picked up and put into LK medium containing 60 μg/mL ampicillin. The cultures of transformants were grown overnight in an incubator at 35° C.

Isolation of Plasmid pAQ-EX1 from KNabc

In an experiment, pAQ-Ex1 was isolated from E. coli KNabc using QIAgen Spin Miniprep Kit. In an experiment, the following results for Sample 1 were obtained: OD260 (1.088), OD280 (0.564), OD260/OD280 (1.93), concentration (54 ng/μL), and OD260/OD230 (1.94). In an experiment, the following results for Sample 2 were obtained: OD260 (1.287), OD280 (0.687), OD260/OD280 (1.87), concentration (64 ng/μL), and OD260/OD230 (1.76).

Amplification of atfA from Acinetobacter

In an experiment, the master solution was based on using 20 μL reaction solution per tube. Each reaction utilized 2 μL forward primer, 2 μL reverse primer, 2 μL buffer, 2 μL dNTPs, 0.2 μL Taq, and 1.8 μL water. In an experiment, atfA primers were used with 1 μL (10 ng DNA), 4 μL (40 ng DNA), 6 μL (60 ng DNA), 10 μL (100 ng DNA), and 0 μL (control). In an experiment, atfA primers were also used with 1 μL (1 ng DNA), 4 μL (4 ng DNA), 6 μL (6 ng DNA), and 10 μL (10 ng DNA). The PCR program was as follows: 95° C. for 5 min, 90° C. for 1 min, 30 sec, 55° C. for 2 min, 72° C. for 2 min. After 35 cycles, samples were heated to 72° C. for 10 min, and stored at 4° C.

Transformation of E. coli with pAQ-EX1

In an experiment, cells were collected from induced competent cells. Cells were placed in ice water for 10 min. 50 ng DNA was added and the cells were mixed with a slight shaking of the hand. The cells were placed into ice water again for 30 min. The cells were placed in 42° C. water for exactly 90 sec without shaking and then rapidly immersed into ice water for 2-3 min. 800 μL LK liquid medium was added to the cells, which were then put into a 37° C. water bath for 1 hr with shaking. Cycled water was used as power to shake the tube. 20 μL or 40 μL of transformed culture were used to spread the plates, which were placed in an incubator at 35° C.

### Plating Experiments on Synechococcus Strains

In an experiment, 5 strains of Synechococcus were used (UTEX 2389, 2390, 2434, 2537 and syn5). The cell numbers were counted before cells were plated on plates using a hamaetometer. Strain UTEX 2389 was plated at 6.25×10⁷ cells/μL using medium B3N. Strain UTEX 2390 was plated at 5.7×10⁷ cells/μL using medium B3N. Strain UTEX 2537 was plated at 8.8×10⁷ cells/μL using medium B3N. Strain UTEX 2434 was plated at 2.3×10⁷ cells/μL using medium MES. Strain Syn5 was plated at 5.8×10⁷ cells/μL using medium BG-11-1. For strains UTEX 2389, UTEX 2390, UTEX 2537 and syn5, the following volumes of solution were used for plating: 1 μL, 4 μL or 10 μL. For strain UTEX 2434, the following volumes of solution were used for plating: 3 μL, 6 μL, or 30 Plates were kept in the dark overnight and then in dim light for 3 d. The plates were subsequently placed in light of 40-50 μE.

### Primers Design for Amplification of PDC, AdhB and AtfA Genes

In an experiment, keeping pAQ-EX1 in mind, restriction sites for all pdc, adhB and atfA genes were examined. The restriction sites did not cut all 3 genes. Using this information, primers were designed as follows: (1) PDC forward (XXX-BamHI) and PDC reverse (XXX-PvuI); (2) AdhB forward (XXX-PvuI) and AdhB reverse (XXXX-mal), and (3) AtfA forward (XXX-XmaI) and AtfA reverse (XXX-BamHI). BamHI has only one site on pAQ-EX1.

Primer Designs for Amplifications of 3 Genes from Bacteria

In an experiment, the PDC forward primer was (BamHI) GTCGGATCCTGATTCAGACATAGTGT (SEQ ID NO: 12) and the PDC reverse primer was (PvuI) CATCGATCGCCTTAAGCTCTAAGT (SEQ ID NO: 13). In an experiment, the AdhB forward primer was (PvuI) CATCGATCGGGTTGTTGCTTTAAACT (SEQ ID NO: 14) and the AdhB reverse primer was (XmaI) GATCCCGGGACGGAAAACCGTTTTCC (SEQ ID NO: 15). In an experiment, the AtfA forward primer was (XmaI) GATCCCGGGTTATTAATATCTTTGCAG (SEQ ID NO: 16) and the AtfA reverse primer was (BamHI) GTCGGATCCTTTAGTTTTATCTGAT (SEQ ID NO: 17).

Isolation of DNA from Bacteria

In an experiments, the lysis buffer comprised 3% CTAB, 1% SDS, 100 mM Tris pH 8.0, 100 nM EDTA, 1.4 M NaCl, 2 μL Proteinase K (20 mg/mL). The phenol component comprised phenol, chloroform, and isoamyl in a 25:24:1 ratio, respectively. Bacterial cells were collected by centrifuge and washed in sterile distilled water 3 times. The cells were then centrifuged and placed in a 60° C. water bath for 3-40 min. 500 μL lysis buffer was added and the temperature was kept at 60° for 40 min. 800 μL phenol was added to the cells, which were mixed thoroughly, and then centrifuged at 5,000 rpm for 5 min. The supernatant was taken and 800 μl of the phenol:chloroform:isoamyl solution was added to the supernatant and mixed thoroughly. The solution was allowed to sit for 15 min before being centrifuged at 5,000 rpm. The supernatant was again taken and 800 μL of the phenol:chloroform:isoamyl solution was again added and mixed thoroughly. The solution was allowed to sit for 15 min before being centrifuged again at 5,000 rpm. The supernatant was taken and 800 μL chloroform was added and mixed thoroughly. The solution was centrifuged at 5,000 rpm for 5 min. This chloroform step was repeated. The supernatant was taken and twice the volume of ethanol (70%) was added and the solution was kept at 4° C. for at least 1 hr. The solution was centrifuged at 12,000 rpm for 15 min and the supernatant was discarded. The remainder was dried at room temperature or in a vacuum. 100 μL sterile water was added and mixed thoroughly. In an experiment, the measurement of OD260:0 D280 was taken and the ratio was calculated. In an experiment, the following results were generated for Zymononas: OD260 (90.028), OD280 (50.456), OD260/OD280 (1.78), concentration (4500 ng/μL), and OD260/OD230 (1.85). In an experiment, the following results were generated for Acinetobacter: OD260 (5.862), OD280 (3.101), OD260/OD280 (1.89), concentration (293 ng/μL), and OD260/OD230 (2.12).

Amplification of 3 Genes from Zymomonas and Acintobacter

In an experiment, the genes pdc and adhB were amplified from Zymomonas and atfA was amplified from Acinetobacter. Primers were dissolved and diluted to 20 uM. Genomic DNA from previous extractions was diluted to 10 ng/μL. A master solution was prepared based on 20 μL reaction solution per tube. In an experiment, forward primer (2 μL), reverse primer (2 μL) buffer (2 μL), dNTPs (2 μL), Taq (0.2 μL), and water (1.8 μL were used for each reaction. For reactions using pdc primers, the following amounts were used: (1) PDC 1 μL (10 ng DNA), (2) PDC 4 μL (40 ng DNA), (3) PDC 6 μL (60 ng DNA), (4) PDC 10 μL (100 ng DNA), (5) 0 μL (control). For reactions using atfA primers, the following amounts were used: (1) atfA 1 tit (10 ng DNA), (2) atfA 4 μl, (40 ng DNA), (3) atfA 6 μL (60 ng DNA), (4) atfA 10 μL (100 ng DNA), (5) 0 μL (control). For reactions using adhB primers, the following amounts were used: (1) adhB 1 μL (10 ng DNA), (2) adhB 4 μl, (40 ng DNA), (3) adhB 6 (60 ng DNA), (4) adhB 10 μL (100 ng DNA), and (5) 0 μL (control). In an experiment, the PCR program for these reactions was as follows: 95° C. for 5 min, 90° C. for 1 min, 30 sec, 48° C. for 2 min, 72° C. for 2 min, which sequences was repeated 5 times. The program then continued with 90° C. for 1 min, 30 sec, 52° C. for 2 min, 72° C. for 2 min, which sequence was repeated 30 times. The samples were then heated to 72° C. for 10 min and stored at 4° C.

### Purification of PCR Products

In an experiment, the pdc and adhB PCR products were purified using enElute PCR Clean-up Kit (Sigma), and the following results were generated for pdc: OD260 (2.274), OD280 (1.224), OD260/OD280 (1.86), concentration (113.7), and OD260/OD230 (1.89). In an experiment, the following results were generated for adh: OD260 (2.523), OD280 (1.353), OD260/OD280 (1.86), concentration (126.2 ng/μL), and OD260/OD230 (1.96). The products were then sequenced.

The plasmid pAQ-EX1 can be used with the methods disclosed herein. (see FIG. 1). The complete amino acid sequence for this expression vector is listed in SEQ ID NO. 8, and the complete nucleotide sequence for this expression vector is listed in SEQ ID NO. 9. The plasmid pAQ-Ex1 carries four unique restriction sites in MCS: BamHI, SmaI, pstl, and Kpnl. For example, in some aspects of the invention, three genes are inserted downstream of the rbcL promoter (the large subunit of ribulose-1,5-bisphosphate carboxylase/oxygenase) located in MCS. First, pdc amplified from genomic DNA 40 bp upstream and 29 bp downstream of pdc gene, and the PCR product contains pstl and pvuI sites. Second, adhB amplified from genomic DNA 43 bp upstream and 27 bp downstream of adhB gene; and the PCR product contains pvuI and BamHI sites. Third, atfA amplified from genomic DNA 21 bp upstream and 130 bp downstream of atfA gene, and the PCR product contains BamHI and XmaI sites. (FIG. 2).

The following transformation protocol can be used. First, the cell were cultured in 100 mL of BG-11 liquid medium at 28° C. under cool white fluorescent light and subcultured at mid-exponential phase of growth. To 1.0 mL of cell suspension containing 2×10⁸ cells (for Synechoccus sp. PCC 7942, A₇₃₀=0.5) of 10⁹ cells (for Synechoccus sp. PCC 6301, A₇₃₀=2.5), which were cultured at the mid-exponential phase of growth, 0.5 or 1.0 μg of donor DNA (in 10 mM Tris/1 mM EDTA, pH8.0) was added, and the mixture was incubated in the dark at 26° C. overnight. After incubation for further 6 hours in the light, the transformants were directly selected on BG-11 agar plates containing 1.5% agar, 1 mM sodium thiosulfate and 0.5 μg/mL Ap. The transformants frequency was calculated by counting the number of transformants after 15 days.

SEQUENCES

```
SEQ ID NO. 1
Nucleotide sequence for homo sapies alpha-beta hydrolase
domain containing 1 (ABHD1)
      1 gcggggaccg gacctgcaca ggccgcctat ggcgggcggc gggtgggacc gcgagttaca
    61 gccggccaac tggggccagc caggagcctg agggtcggaa gcccccaaca caagatgctg
  121 agctccttcc tgagccccca gaatggcacc tgggcagaca ccttctctct gctcttggct
  181 cttgccgttg ccctctactt gggctactac tgggcatgtg tgcttcagag gcctcggctg
  241 gtggctgggc cgcagtttct ggccttcctg gagccacact gttccatcac caccgagact
  301 ttctacccaa cgctgtggtg ttttgagggg cgactacaaa gcatcttcca agtcctcctg
  361 cagtctcagc ccctagtcct ttatcagagt gacatcctcc aaacaccaga tggaggccag
  421 ctcctgctag actgggccaa gcagcctgac agcagccaag accctgatcc tactacccag
  481 cccattgtgc tgctgcttcc tggcatcact ggcagtagcc aggagacata cgtcttgcac
  541 ctagttaacc aagctctgag ggatggctac caggctgtcg tgtttaacaa ccggggctgc
  601 cgtggggagg aactgcggac ccacagggct ttttgtgcca gcaatactga agatctagag
  661 acagtcgtga accacataaa gcatcgttat ccccaagctc cactgctggc cgtgggcatc
  721 tcttttggag ggatactggt gctgaatcac ctggcacagg ccaggcaggc tgcagggctg
  781 gtggcagcac tgactctgtc tgcatgctgg gattcctttg agaccactcg ctccctggaa
  841 accccactca actcactgct cttcaatcag cccctcactg ctgggctctg ccaacttgtg
  901 gaacgaaaca gaaaggtgat tgaaaaggtg gtggacatag actttgtact acaggcccgt
  961 acaatccgcc agtttgatga gcgctacaca tctgtggcct ttggatatca agactgtgtt
1021 acctactaca aagcagcaag ccctagaacc aagatagatg ccatccggat ccctgtgctc
1081 tatctcagtg cagcagatga ccccttctcc cccgtctgtg cccttcccat acaggccgcc
1141 caacactccc cctacgttgc gctgctcatc acagcccggg gtggccacat cggcttcctg
1201 gaagggctgc tcccgtggca gcactggtac atgagccgcc tcttgcatca gtacgccaaa
1261 gccatcttcc aggacccaga ggggctgcct gacctcaggg ctctcttacc ttctgaggac
1321 agaaacagct gacaagagta ccatttgggg tctcagttca ctctttcctt gtttattaaa
1381 tatcaacttt tcctgcctaa aaaaaaaaaa aaaaaaaaaa aaaaaaaaaa aaaaaaaaaa
1441 aaaaaaaaaa aaaaaaaaaa aaaaaaaaaa aaaa
SEQ ID NO. 2
Nucleotide sequence for homo sapies alpha-beta hydrolase
domain containing 3 (ABHD3)
      1 gggagtaggc agcggcgccg cgtccgctct cgcccgctct cgcccgctcg ccagccggct
    61 ctcctcccgc cgcaggaccc gcgcgccgcg ctcgggggcc atgcagcgcc tggccatgga
  121 cctgcggatg ttgtcccggg agctctccct ctacctggaa caccaagtcc gggtggggtt
  181 cttcggctcg ggggtgggct tatcccttat cctgggcttc agcgtcgctt atgccttcta
  241 ctacctgagc agcattgcca agaaacccca gttagtgacc gggggtgaga gtttcagccg
  301 cttccttcaa gaccactgtc ccgtggttac agaaacgtac tacccgacgg tctggtgctg
  361 ggagggtcga ggacagaccc tgcttagacc tttcatcact tcgaagcccc cggtgcagta
  421 caggaatgaa cttattaaaa ctgcagatgg aggacagatt tcactggact ggtttgataa
  481 tgataacagt acgtgttata tggatgccag caccagacct actatcttat tgttgcctgg
  541 cctcacggga acaagcaagg agtcatatat ccttcatatg atccatctta gtgaagaatt
  601 aggatacaga tgtgtggttt ttaacaacag aggagtggcg ggggagaatc tcttgacgcc
  661 aaggacttat tgttgtgcta acactgaaga cttggagaca gttattcacc atgtacacag
  721 cctgtaccct tctgctcctt tcctggcagc aggggtttca atgggaggaa tgctgcttct
  781 aaattacttg ggcaaaattg ggtccaaaac gcctttgatg gcagctgcaa ctttttccgt
  841 tggttggaac accttcgctt gctcagagtc attggaaaaa ccactgaact ggctactttt
  901 taattactat ttgacaacct gccttcagtc ttcagttaat aagcaccgac atatgtttgt
  961 aaaacaagtt gatatggatc atgtcatgaa ggctaaatcc atcagagagt ttgataagcg
1021 attcacttca gtcatgtttg gataccaaac aattgatgat tattatactg atgccagtcc
1081 gagtcctaga ctgaagtcag taggaattcc agtattgtgt ctaaattctg tggatgatgt
1141 tttctcaccc agtcatgcta ttccaataga aactgctaag caaaatccta atgttgcttt
1201 ggtccttact tcttatggag gccatattgg ttttctggag ggaatctggc caagacagtc
1261 cacttacatg gatcgtgtct tcaagcaatt tgtgcaagcc atggttgagc atggacatga
1321 actctcttaa catgtagttc tttgggtgca ttttgtctga accacaattg tgaaggcagc
1381 tcagcttagt gcacaaattt taactgttgt atataaagca aataagccag cagatgggtg
1441 aagaggtcca gaatgatatg caaaaactac tttttagaga aacaaaacaa ctttgtagca
1501 acaaattaaa tatagtatta gattgttact tacgtagatt ttatttttac tatgccttac
1561 caagtacatc cttaaacaaa gtagtatgta catgaaattg cacttaacca aaactattgt
1621 gtaaaacaaa ttttaattcc tcagggtttt aatttaaact agtatttttt tagattattt
1681 gttttaggtg atttaatggt actttaataa ctactaagaa atattggcta tttcaatgta
1741 agttataagg tggtacattc ctaagggtat ttatagttga tgataacatg aaaactgaaa
1801 taagataaaa tacaacgtgc taaatctttt atgtattcta actttaaaag acaagtgcaa
1861 caaagttaga ctgacttcta tatgtgctct tttactctga taatattaaa ttaggactaa
1921 cttatgtttt ataatgatta taatttacat gcttattttt aaaatagtat atgtggacac
1981 atatatatca ttatattaaa ataaattcta ccattttaaa ttggaaaaaa aaaaaaaaaa
SEQ ID NO. 3
Amino acid sequence for homo sapies alpha-beta
hydrolase domain containing 1 (ABHD1)
Accession number 24660060
MLSSFLSPQNGTWADTFSLLLALAVALYLGYYWACVLQRPRLVAGPQFLAFLEPHC
SITTETFYPTLWCFEGRLQSIFQVLLQSQPLVLYQSDILQTPDGGQLLLDWAKQPDSS
QDPDPTTQPIVLLLPGITGSSQETYVLHLVNQALRDGYQAVVFNNRGCRGEELRTHR
AFCASNTEDLETVVNHIKHRYPQAPLLAVGISFGGILVLNHLAQARQAAGLVAALTL
SACWDSFETTRSLETPLNSLLFNQPLTAGLCQLVERNRKVIEKVVDIDFVLQARTIRQ
FDERYTSVAFGYQDCVTYYKAASPRTKIDAIRIPVLYLSAADDPFSPVCALPIQAAQH
SPYVALLITARGGHIGFLEGLLPWQHWYMSRLLHQYAKAIFQDPEGLPDLRALLPSE
DRNS
SEQ ID NO. 4
Amino acid sequence for homo sapies alpha-beta hydrolase
domain containing 3 (ABHD3)
Accession number 34304337
MQRLAMDLRMLSRELSLYLEHQVRVGFFGSGVGLSLILGFSVAYAFYYLSSIAKKPQ
LVTGGESFSRFLQDHCPVVTETYYPTVWCWEGRGQTLLRPFITSKPPVQYRNELIKTA
DGGQISLDWFDNDNSTCYMDASTRPTILLLPGLTGTSKESYILHMIHLSEELGYRCVV
FNNRGVAGENLLTPRTYCCANTEDLETVIHHVHSLYPSAPFLAAGVSMGGMLLLNY
LGKIGSKTPLMAAATFSVGWNTFACSESLEKPLNWLLFNYYLTTCLQSSVNKHRHM
FVKQVDMDHVMKAKSIREFDKRFTSVMFGYQTIDDYYTDASPSPRLKSVGIPVLCLN
SVDDVFSPSHAIPIETAKQNPNVALVLTSYGGHIGFLEGIWPRQSTYMDRVFKQFVQA
MVEHGHELS
SEQ ID NO. 5
Amino acid sequence for Z. mobilis alcohol dehydrogenase b (ADH B)
Accession number BAF76066
MASSTFYIPF VNEMGEGSLE KAIKDLNGSG FKNALIVSDA FMNKSGVVKQ
VADLLKAQGI NSAVYDGVMP NPTVTAVLEG LKILKDNNSD FVISLGGGSP
HDCAKAIALV ATNGGEVKDY EGIDKSKKPA LPLMSINTTA GTASEMTRFC
IITDEVRHVK MAIVDRHVTP MVSVNDPLLM VGMPKGLTAA TGMDALTHAF
EAYSSTAATP ITDACALKAA SMIAKNLKTA CDNGKDMPAR EAMAYAQFLA
GMAFNNASLG YVHAMAHQLG GYYNLPHGVC NAVLLPHVLA YNASVVAGRL
KDVGVAMGLD IANLGDKEGA EATIQAVRDL AASIGIPANL TELGAKKEDV
PLLADHALKD ACALTNPRQG DQKEVEELFL SAF
SEQ ID NO. 6
Amino acid sequence for Z. mobilis pyruvate decarboxylase (PDC)
Accession number AAA27697
MSYTVGTYLAE RLVQIGLKHH FAVAGDYNLV LLDNLLLNKN MEQVYCCNEL
NCGFSAEGYA RAKGAAAAVV TYSVGALSAF DAIGGAYAEN LPVILISGAP
NNNDHAAGHV LHHALGKTDY HYQLEMAKNI TAAAEAIYTP EEAPAKIDHV
IKTALREKKP VYLEIACN IASMPCAAP GPASALFN DEASDEA SLNAAVEE
TLKFIANRD KVAVLVGS KLRAAGAEEA AVKFADALG GAVATMAAA
KSFFPEENPH YIGTSWGEVSY PGVEKTMKE ADAVIALAP VFNDYSTTGW
TDIPDPKKLVLA EPRSVVVNGI RFPSVHLKDY LTRLAQKVSKK TGALDFFK
SLNAGELKK AAPADPSA PLVNAEIAR QVEALLTPN TTVIAETGDSW FNAQRMKLP
NGARVEYEM QWGHIGWSVP AAFGYAVGAP ERRNILMVGD GSFQLTAQEV
AQMVRLKLP VIIFLINNYG YTIEVMEHDGP YNNIKNWDY AGLMEVFNG
NGGYDSGAGK GLKAKTGGE LAEAIKVALA NTDGPTLIE CFIGREDCTE
ELVKWGKRVA AANSRKPVNKLL
SEQ ID NO. 7
Amino acid sequence for Acinetobacter sp. strain
ADP1 wax ester synthase/acyl-CoA:
diacylglycerol acyltransferase (WS/DGAT)
Accession number AF529086
MRPLHPIDFI FLSLEKRQQP MHVGGLFLFQ IPDNAPDTFI QDLVNDIRIS
KSIPVPPFNN KLNGLFWDED EEFDLDHHFR HIALPHPGRI RELLIYISQE
HSTLLDRAKP LWTCNIIEGI EGNRFAMYFK IHHAMVDGVA GMRLIEKSLS
HDVTEKSIVP PWCVEGKRAK RLREPKTGKI KKIMSGIKSQ LQATPTVIQE
LSQTVFKDIG RNPDHVSSFQ APCSILNQRV SSSRRFAAQS FDLDRFRNIA
KSLNVTINDV VLAVCSGALR AYLMSHNSLP SKPLIAMVPA SIRNDDSDVS
NRITMILANL ATHKDDPLQR LEIIRRSVQN SKQRFKRMTS DQILNYSAVV
YGPAGLNIIS GMMPKRQAFN LVISNVPGPR EPLYWNGAKL DALYPASIVL
DGQALNITMT SYLDKLEVGL LACRNALPRM QNLLTHLEEE IQLFEGVIAK
QEDIKTAN
SEQ ID NO. 8
Amino acid sequence for Expression vector pAQ-EX1 DNA
Accession number AB071392
MTPYEYLLYSDEPALRRNDGRLRDTWLKRYAFVEHGGWWCSGIDIKTGKDSLWGC
FKGDRPRKDREDKKPIKYEHPPRVATEIFTLKVDRGTWRKIAKRHKVELPETDQGFW
EWVLAHPELPIIITEGAKKAGALLTAGYCAIGLPGIYNGYRTPKNDHGEPMRQLRHLI
PELDLLAKNNRAIAFCFDQDKKPKTIKAVNGAIQTTGALLEKAGAKVSVITWHQDA
KGVDDLIVEHGAKALHNRYKHRKPLAVWEMDNLTDITTQVDLTVDQRYLDIDPRAI
PKDAQIIFIKSAKGTGKTEWLGKIVKLAQDDCARVLVLTHRIQLAKELARRLDIDHIS
ELDSSPTGGALGMAMCIDSLHPDSQAHFNFMEWHGAHIVLDEIEQVLGHALGSSTCT
QDRAKILETFYNLILYALRTGGKLYCSDADLSPISYELIKYILDGCEFKPFTILNTYKPC
LEQQRDLFFYEGNDPRDLLTNLRQAIENGEKTLVFTAAQKTASTYSTQNLESLFREK
YPDKRILRIDAESVAEPGHPAYGCIDSLNAILPLYDIVLCSPAVETGVSIDIKDHFDSV
WGMGSGVQTVNGFCQGLERLRDNVPRHVWIPKFSPHSNRIANGGYTAKAIARDQHR
YAELTHKLIGEHAAECSGLEDSLKPFLWAYCRYAALANRGFGSYREAILNKLLSEGY
VQKDLSEIDPALAKDYRDELKAVKDHNYLQERVAISKVENPDDRQYEKLKRQRAKS
ETERHQERHGKLSRSYGLTVTPELVEKDDDGWYSQLQLEYYLTVGKAFCSARDRAK
YDQLQHEGFVFKPDINRRSLSPKIHLLELLNIHQFLKPGVTFTGASLEGFKENCLRYA
KPIKWILGRTITDKMSPLEIAQALLGKLDRKLEYKGRFGSRDNRQRVYEAIAPNDQRE
KVFAHWLQRDQAKLGAVSNPCINRFIQEA
SEQ ID NO. 9
Nucleotide sequence for Expression vector pAQ-EX1 DNA
      1 tcgcgcgttt cggtgatgac ggtgaaaacc tctgacacat gcagctcccg gagacggtca
    61 cagcttgtct gtaagcggat gccgggagca gacaagcccg tcagggcgcg tcagcgggtg
  121 ttggcgggtg tcggggctgg cttaactatg cggcatcaga gcagattgta ctgagagtgc
  181 accatatgcg gtgtgaaata ccgcacagat gcgtaaggag aaaataccgc atcaggcgcc
  241 attcgccatt caggctgcgc aactgttggg aagggcgatc ggtgcgggcc tcttcgctat
  301 tacgccagct ggcgaaaggg ggatgtgctg caaggcgatt aagttgggta acgccagggt
  361 tttcccagtc acgacgttgt aaaacgacgg ccagtgaatt tctagaattc gagcgggatt
  421 ttatggcttt tttaggtatt tttgtaaggg taaaataggc ccatcaaaca gcattagaaa
  481 tgctaatcag cccaaaaaac aaaagcaatc tttttttgtt gctaaaagat aaaaataagt
  541 cgaggctgtg gtaacatatc ccacagatta aagaaagtca taagacttga atcttcagaa
  601 ttttaaaaag cagttttgcc aacgtaagat ttttgaagtt ttcgaccaac aataccgtta
  661 ctggtatttg tctgttaaag ataagcattt ttgctggagg aaaaccccat gctagactgc
  721 agggatcccg ggtaccatcg atgaattcga gctcatgccc ccggtagtgc cctcaccctc
  781 agtgaggctt atgatctggc acagtctagg ggctaccaga agagcaaagg cgctttccgc
  841 aagggcataa aacgtcccga taccgcagag caaatcttga gattgtttgg gattgccaag
  901 ggagagctgc acaacgagta tttcgatcac gggtctaagc ctcctgaata aatctattta
  961 tacaggggtt ggacacggcc cctaattttg cttggtcacg ctgtaaccaa tgagcaaaga
1021 ccttttcgcg ctgatcgtta ggggcgatcg cctcatagac ccgctgacgg ttatcccgcg
1081 atccaaagcg ccccttgtat tccaatttcc ggtcaagctt gcctaggagc gcctgagcaa
1141 tttcgagcgg gctcattttg tcggtgatcg ttctgccaag aatccacttg atcggcttgg
1201 cataccgcaa acaattttcc ttgaaccctt caaggctcgc cccggtgaat gtcacccctg
1261 gttttaagaa ctgatggatg ttgagtagct ctaacaggtg aatctttggt gagagcgatc
1321 gcctgttgat atccggctta aatacaaagc cctcatgttg gagctggtca tatttcgccc
1381 ggtcgcgggc agagcagaat gctttcccaa cggttaagta gtattcgagc tggagctgag
1441 agtaccaccc atcatcatct ttctcgacaa gctcaggggt cacagttaac ccataggagc
1501 gagaaagttt cccgtggcgt tcttggtgcc gttccgtctc agatttcgcc cgctgacgct
1561 tcagtttttc gtactggcga tcgtcaggat tttctacttt agaaatcgca accctttcct
1621 gtagataatt atggtctttc accgctttta attcgtctcg ataatcctta gccaatgctg
1681 gatcgatttc gctcaaatct ttctgtacat agccctcaga aagcagctta tttaaaatcg
1741 cttcccggta actgccaaag ccacggttag caagcgccgc atagcgacaa taggcccaaa
1801 ggaatggttt taaagaatct tctaacccac tgcattcagc ggcgtgctca ccgattaatt
1861 tgtgggtgag ctctgcatag cggtgctggt cacgggcgat cgccttagcg gtgtagcccc
1921 cattggcgat ccggttcgag tgtggggaaa atttcggtat ccaaacatgg cgagggacgt
1981 tatcccgtaa ccgctctagc ccttggcaga aaccgttaac ggtctgaacc cctgaaccca
2041 tgccccaaac agaatcaaaa tgatccttga tatcgatact caccccggtc tcgacagcgg
2101 gcgagcagag cacaatatcg tacaggggca aaattgcatt cagcgagtca atacaaccat
2161 aggcagggtg ccccggttca gcgaccgatt cagcatcgat tctcaagatt ctcttatcgg
2221 gatatttctc cctaaagagt gattctaagt tctgcgtgct gtaggtcgat gcggtctttt
2281 gggcagcggt aaagacaagt gttttctcac cgttctcaat cgcttgtctg agattggtta
2341 acaggtctct agggtcattc ccttcataga aaaacaggtc tctttgttgc tctaaacagg
2401 gcttataggt attcagaatg gtgaatggtt tgaactcaca accgtcaaga atgtacttga
2461 ttagctcata ggagatggga gataaatcag catcagagca gtagagtttt ccgcctgtcc
2521 ttagggcata aaggattagg ttgtagaacg tttcaaggat tttcgcccgg tcttgggtac
2581 aggtcgagct acccaaagcg tgccctaaaa cttgctcgat ttcgtctagg acaatgtgag
2641 cgccgtgcca ttccatgaaa ttaaaatgag cttggctatc gggatgtagg ctatcgatac
2701 acatcgccat ccctagagcg cccccggtcg ggctactgtc gagctcgcta atgtgatcga
2761 tatcgagacg gcgggcgagc tccttggcta attggatgcg gtgagtcaaa accagtacgc
2821 gagcgcaatc atcttgggcg agcttaacga ttttccctaa ccattctgtt ttcccggtgc
2881 ccttggcaga tttaatgaaa ataatctgag catccttggg gatagcacgc ggatcgatgt
2941 cgagatagcg ctgatcgacc gttagatcga cttgcgtggt gatatcggtg agattatcca
3001 tctcccagac tgctaagggc ttgcggtgct tgtagcggtt atggagtgct ttcgctccgt
3061 gctcgacgat cagatcatca acacctttcg cgtcctggtg ccaggtgatc accgatactt
3121 tcgccccggc cttctctagg agtgccccgg tagtttggat cgccccgttc actgccttga
3181 tcgtcttggg tttcttgtct tggtcaaaac agaaggcgat cgcccggtta tttttcgcca
3241 acaggtcaag ctctggaatg aggtgccgta gctgtcgcat tggctcgcca tggtcatttt
3301 ttggcgttct gtagccgttg taaatccccg gtagaccgat ggcgcaataa ccagcggtta
3361 ggagggcccc tgccttcttc gcgccctcag tgatgatgat cggtagctca ggatgggcta
3421 gtacccattc ccagaagcct tgatcggttt ctggtagctc gactttgtgg cgcttggcaa
3481 tcttgcgcca ggtgccccgg tctaccttga gagtgaaaat ttcggtggct acccgtggcg
3541 ggtgctcata cttgatcggt ttcttatctt cgcgatcttt acgggggcga tcgcctttga
3601 agcaacccca aagagaatct ttcccggtct taatatcgat gcctgagcac caccaaccgc
3661 catgctcgac aaaggcatat cgcttaagcc aagtatcccg aagcctgcca tcattgcggc
3721 gtaatgctgg ctcatcactg tagagcaggt attcataggg cgtcatccct gacaacgatc
3781 taacatttaa ccgggcaatc tcaggggcga ccccggaacc ctctacccat tccctatagt
3841 gagattctgc gataaaatga gaacggttct cactgacgct actcgctaaa tctgccccgg
3901 tattactgct agatgtaaaa ttatctgata cgatcacagt gagttaagga gcttggcgta
3961 atcatggtca tagctgtttc ctgtgtgaaa ttgttatccg ctcacaattc cacacaacat
4021 acgagccgga agcataaagt gtaaagcctg gggtgcctaa tgagtgagct aactcacatt
4081 aattgcgttg cgctcactgc ccgctttcca gtcgggaaac ctgtcgtgcc agctgcatta
4141 atgaatcggc caacgcgcgg ggagaggcgg tttgcgtatt gggcgctctt ccgcttcctc
4201 gctcactgac tcgctgcgct cggtcgttcg gctgcggcga gcggtatcag ctcactcaaa
4261 ggcggtaata cggttatcca cagaatcagg ggataacgca ggaaagaaca tgtgagcaaa
4321 aggccagcaa aaggccagga accgtaaaaa ggccgcgttg ctggcgtttt tccataggct
4381 ccgcccccct gacgagcatc acaaaaatcg acgctcaagt cagaggtggc gaaacccgac
4441 aggactataa agataccagg cgtttccccc tggaagctcc ctcgtgcgct ctcctgttcc
4501 gaccctgccg cttaccggat acctgtccgc ctttctccct tcgggaagcg tggcgctttc
4561 tcatagctca cgctgtaggt atctcagttc ggtgtaggtc gttcgctcca agctgggctg
4621 tgtgcacgaa ccccccgttc agcccgaccg ctgcgcctta tccggtaact atcgtcttga
4681 gtccaacccg gtaagacacg acttatcgcc actggcagca gccactggta acaggattag
4741 cagagcgagg tatgtaggcg gtgctacaga gttcttgaag tggtggccta actacggcta
4801 cactagaagg acagtatttg gtatctgcgc tctgctgaag ccagttacct tcggaaaaag
4861 agttggtagc tcttgatccg gcaaacaaac caccgctggt agcggtggtt tttttgtttg
4921 caagcagcag attacgcgca gaaaaaaagg atctcaagaa gatcctttga tcttttctac
4981 ggggtctgac gctcagtgga acgaaaactc acgttaaggg attttggtca tgagattatc
5041 aaaaaggatc ttcacctaga tccttttaaa ttaaaaatga agttttaaat caatctaaag
5101 tatatatgag taaacttggt ctgacagtta ccaatgctta atcagtgagg cacctatctc
5161 agcgatctgt ctatttcgtt catccatagt tgcctgactc cccgtcgtgt agataactac
5221 gatacgggag ggcttaccat ctggccccag tgctgcaatg ataccgcgag acccacgctc
5281 accggctcca gatttatcag caataaacca gccagccgga agggccgagc gcagaagtgg
5341 tcctgcaact ttatccgcct ccatccagtc tattaattgt tgccgggaag ctagagtaag
5401 tagttcgcca gttaatagtt tgcgcaacgt tgttgccatt gctacaggca tcgtggtgtc
5461 acgctcgtcg tttggtatgg cttcattcag ctccggttcc caacgatcaa ggcgagttac
5521 atgatccccc atgttgtgca aaaaagcggt tagctccttc ggtcctccga tcgttgtcag
5581 aagtaagttg gccgcagtgt tatcactcat ggttatggca gcactgcata attctcttac
5641 tgtcatgcca tccgtaagat gcttttctgt gactggtgag tactcaacca agtcattctg
5701 agaatagtgt atgcggcgac cgagttgctc ttgcccggcg tcaatacggg ataataccgc
5761 gccacatagc agaactttaa aagtgctcat cattggaaaa cgttcttcgg ggcgaaaact
5821 ctcaaggatc ttaccgctgt tgagatccag ttcgatgtaa cccactcgtg cacccaactg
5881 atcttcagca tcttttactt tcaccagcgt ttctgggtga gcaaaaacag gaaggcaaaa
5941 tgccgcaaaa aagggaataa gggcgacacg gaaatgttga atactcatac tcttcctttt
6001 tcaatattat tgaagcattt atcagggtta ttgtctcatg agcggataca tatttgaatg
6061 tatttagaaa aataaacaaa taggggttcc gcgcacattt ccccgaaaag tgccacctga
6121 cgtctaagaa accattatta tcatgacatt aacctataaa aataggcgta tcacgaggcc
6181 ctttcgtc
SEQ ID NO: 10
GATCCCGGGCAATCCACGCTATGCGCCCATTACATC
SEQ ID NO: 11
GTCGGATCCAGATCACGACTGCAATGGTTCATC
SEQ ID NO: 12
GTCGGATCCTGATTCAGACATAGTGT
SEQ ID NO: 13
CATCGATCGCCTTAAGCTCTAAGT
SEQ ID NO: 14
CATCGATCGGGTTGTTGCTTTAAACT
SEQ ID NO: 15
GATCCCGGGACGGAAAACCGTTTTCC
SEQ ID NO: 16
GATCCCGGGTTATTAATATCTTTGCAG
SEQ ID NO: 17
GTCGGATCCTTTAGTTTTATCTGAT
```

### REFERENCES

1. Abramson J, Smirnova I, Kasho V, Verner G, Kaback H R, Iwata S. (2003) Structure and mechanism of the lactose permease of Escherichia coli. Science, 301: 610-5.
2. Acsadi G, Dickson G, Love D R, Jani A, Walsh F S, Gurusinghe A, Wolff J A, Davies K E. (1991) Human dystrophin expression in mdx mice after intramuscular injection of DNA constructs. Nature, 352: 815-8.
3. Altschul S F, Lipman D J. (1990) Protein database searches for multiple alignments. Proc Natl Acad Sci USA, 87: 5509-13.
4. Ambudkar S V, Kim I W, Sauna Z E. (2006) The power of the pump: mechanisms of action of P-glycoprotein (ABCB1). Eur J Pharm Sci., 27: 392-400.
5. Ashburner M. Drosophila A Laboratory Handbook, Cold Spring Harbor Laboratory Press, 1989.
6. Bagshawe K D. (1989) The First Bagshawe lecture. Towards generating cytotoxic agents at cancer sites. Br J Cancer, 60: 275-81.
7. Bagshawe K D, Springer C J, Searle F, Antoniw P, Sharma S K, Melton R G, Sherwood R F. (1993) A cytotoxic agent can be generated selectively at cancer sites. Br J Cancer, 58: 700-3.
8. Banerji J, Olson L, Schaffner W. (1983) A lymphocyte-specific cellular enhancer is located downstream of the joining region in immunoglobulin heavy chain genes. Cell, 33: 729-40.
9. Battelli M G, Abbondanza A, Tazzari P L, Bolognesi A, Lemoli R M, Stirpe F. (1992) T lymphocyte killing by a xanthine-oxidase-containing immunotoxin. Cancer Immunol Immunother., 35: 421-5.
10. Berkner K L, Schaffhausen B S, Roberts T M, Sharp P A. (1987) Abundant expression of polyomavirus middle T antigen and dihydrofolate reductase in an adenovirus recombinant. J. Virol., 61: 1213-20.
11. Bird D A, Laposata, M, Hamilton J A. (1996) Binding of ethyl oleate to low density lipoprotein, phopholipid vesicles, and albumin: a ¹³C NMR study. J. Lipid Res., 37: 1449-58.
12. Birren B, Green E D, Klaphoz S (Eds.). Genome Analysis: A Laboratory Manual: Analyzing DNA (Vol 1), Cold Spring Harbor Laboratory, 1997.
13. Bockey D, von Schenck W. (2005) Status report—biodiesel production and marketing in Germany 2005. Berlin, Germany: Union for the Promotion of Oil and Protein Plants (UFOP).
14. Bout A, Perricaudet M, Baskin G, Imler J L, Scholte B J, Pavirani A, Valerio D. (1994) Lung gene therapy: in vivo adenovirus-mediated gene transfer to rhesus monkey airway epithelium. Hum Gene Ther., 5: 3-10.
15. Brigham K L, Meyrick B, Christman B, Berry L C Jr, King G. (1989) Expression of a prokaryotic gene in cultured lung endothelial cells after lipofection with a plasmid vector. Am J Respir Cell Mol. Biol., 1: 95-100.
16. Brown D T, Burlingham B T. (1973) Penetration of host cell membranes by adenovirus 2. J. Virol., 12: 386-96.
17. Brown V I, Greene M I. (1991) Molecular and cellular mechanisms of receptor-mediated endocytosis. DNA Cell Biol., 10: 399-409.
18. Caillaud C, Akli S, Vigne E, Koulakoff A, Perricaudet M, Poenaru L, Kahn A, Berwald-Netter Y. (1993) Adenoviral vector as a gene delivery system into cultured rat neuronal and glial cells. Eur J. Neurosci., 5: 1287-91.
19. Carillo H, Lipman D. (1988) The multiple sequence alignment problem in biology. SIAM J. Appl. Math., 48: 1073-82.
20. Chardonnet Y, Dales S. (1970) Early events in the interaction of adenoviruses with HeLa cells. I. Penetration of type 5 and intracellular release of the DNA genome. Virology, 40: 462-77.
21. Cotter M A, Robertson E S. (1999) Molecular genetic analysis of herpesviruses and their potential use as vectors for gene therapy applications. Curr Opin Mol. Ther., 1: 633-44.
22. Coulson A. (1994) High-performance searching of biosequence databases. Trends Biotechnol., 12: 76-80.
23. Creighton T E, Proteins: Structure and Molecular Properties, W. H. Freeman & Co., San Francisco, pp 79-86 (1983).
24. Davidson D, Hassell J A. (1987) Overproduction of polyomavirus middle T antigen in mammalian cells through the use of an adenovirus vector. J. Virol., 61: 1226-39.
25. Devereux J, Haeberli P, and Smithies O. (1994) A comprehensive set of sequence analysis programs for the VAX. Nucleic Acids Research, 12: 387-95.
26. Dyda F, Furey W, Swaminathan S, Sax M, Farrenkopf B, Jordan F. (1993) Catalytic centers in the thiamin diphosphate dependent enzyme pyruvate decarboxylase at 2.4-A resolution. Biochemistry, 32: 6165-70.
27. Feigner P L, Gadek T R, Holm M, Roman R, Chan H W, Wenz M, Northrop J P, Ringold G M, Danielsen M. (1987) Lipofection: a highly efficient, lipid-mediated DNA-transfection procedure. Proc Natl Acad Sci USA, 84: 7413-17.
28. Fiers W, Contreras R, Haegemann G, Rogiers R, Van de Voorde A, Van Heuverswyn H, Van Herreweghe J, Volckaert G, Ysebaert. (1978). Complete nucleotide sequence of SV40 DNA, Nature, 273: 113-20.
29. Gomez-Foix A M, Coats W S, Baque S, Alam T, Gerard R D, Newgard C B. (1992) Adenovirus-mediated transfer of the muscle glycogen phosphorylase gene into hepatocytes confers altered regulation of glycogen metabolism. J Biol. Chem., 267: 25129-34.
30. Greenaway P J, Oram J D, Downing R G, Patel K. (1982) Human cytomegalovirus DNA: BamHI, EcoRI and PstI restriction endonuclease cleavage maps. Gene, 18: 355-60.
31. Grunewald M, Kanner B I. (2000) The accessibility of a novel reentrant loop of the glutamate transporter GLT-1 is restricted by its substrate. J Biol. Chem., 275: 9684-89.
32. Guan L, Nakae T. (2001) Identification of essential charged residues in transmembrane segments of the multidrug transporter MexB of Pseudomonas aeruginosa. J. Bacteriol., 183: 1734-39.
33. Guzman R J, Lemarchand P, Crystal R G, Epstein S E, Finkel T. (1993) Efficient gene transfer into myocardium by direct injection of adenovirus vectors. Circ Res., 73: 1202-07.
34. Haj-Ahmad Y, Graham F L. (1986) Development of a helper-independent human adenovirus vector and its use in the transfer of the herpes simplex virus thymidine kinase gene. J. Virol., 57: 267-74.
35. Hoberg J. Pyruvate Decarboxylase, available at ttp://www.chem.uwec.edu/Webpapers_F98/hoberg/hoberg.html.
36. Hoppner T C, Doelle H W. (1983). Purification and kinetic characteristics of pyruvate decarboxylase and ethanol dehydrogenase from Zymomonas mobilis in relation to ethanol production. Eur. J. Appl. Microbiol. Biotechnol., 17:152-157.
37. Huang Y, Lemieux M J, Song J, Auer M, Wang D N. (2003) Structure and mechanism of the glycerol-3-phosphate transporter from Escherichia coli. Science, 301: 616-20.
38. Hughes B J, Kennel S, Lee R, Huang L. (1989) Monoclonal antibody targeting of liposomes to mouse lung in vivo. Cancer Res., 49: 6214-20.
39. Ingram L O, Conway T, Clark D P, Sewell G W, Preston J F. (1997) Genetic engineering of ethanol production in Escherichia coli. Appl Environ Microbiol., 53: 2420-25.
40. Kalscheuer R, Luftmann H, Steinhachel A. (2004) Synthesis of novel lipids in Saccharomyces cerevisiae by heterologous expression of an unspecific bacterial acyltransferase. Appl Environ Microbiol., 70: 7119-25.
41. Kalscheuer R, Steinbüchel A. (2003) A novel bifunctional wax ester synthase/acyl-CoA:diacylglycerol acyltransferase mediates wax ester and triacylglycerol biosynthesis in Acinetobacter calcoaceticus ADP1. J Biol. Chem., 278: 8075-82.
42. Kalscheuer R, Stolting T, Steinbüchel A. (2006) Microdiesel: Escherichia coli engineered for fuel production. Microbiology, 152: 2529-36.
43. Kinoshita S, Kakizono T, Kadota K, Das K, and Taguchi H. (1985). Purification of two alcohol dehydrogenases from Zymomonas mobilis and their properties. Appl. Microbiol. Biotechnol., 22: 249-254.
44. Kirshenbaum L A, MacLellan W R, Mazur W, French B A, Schneider M D. (1993) Highly efficient gene transfer into adult ventricular myocytes by recombinant adenovirus. J Clin Invest., 92: 381-7.
45. Koronakis V, Sharff A, Koronakis E, Luisi B, Hughes C. (2000) Crystal structure of the bacterial membrane protein TolC central to multidrug efflux and protein export. Nature, 405: 914-9.
46. Krawczyk B, Kur J. (1996) Purification of IHF-like protein from gram-negative bacteria in one chromatographic step. Acta Biochim Pol., 43: 379-82.
47. Laimins L A, Rhoads D B, Epstein W. (1981) Osmotic control of kdp operon expression in Escherichia coli. Proc Natl Acad Sci USA, 78: 464-8.
48. Lanyi J K. (2004) X-ray diffraction of bacteriorhodopsin photocycle intermediates. Mol Membr Biol., 21: 143-50.
49. Lastowski-Perry D, Otto E, Maroni G. (1985) Nucleotide sequence and expression of a Drosophila metallothionein. J Biol. Chem., 260: 1527-30.
50. Le Gal La Salle G, Robert J J, Berrard S, Ridoux V, Stratford-Perricaudet L D,
Perricaudet M, and Mallet J. (1993) An adenovirus vector for gene transfer into neurons and glia in the brain. Science, 259: 988-90.
51. Li X Z, Nikaido H. (2004) Efflux-mediated drug resistance in bacteria. Drugs, 64: 159-204.
52. Litzinger D C, Huang L. (1992) Amphipathic poly(ethylene glycol) 5000-stabilized dioleoylphosphatidylethanolamine liposomes accumulate in spleen. Biochim Biophys Acta., 1127: 249-54.
53. Luecke H. (2000) Atomic resolution structures of bacteriorhodopsin photocycle intermediates: the role of discrete water molecules in the function of this light-driven ion pump. Biochim Biophys Acta., 1460: 133-56.
54. Lusky M, Berg L, Weiher H, Botchan M. (1983) Bovine papilloma virus contains an activator of gene expression at the distal end of the early transcription unit. Mol Cell Biol., 3: 1108-22.
55. Markham P N, Neyfakh A A. (2001) Efflux-mediated drug resistance in Gram-positive bacteria. Curr Opin Microbiol., 4: 509-14.
56. Maruyama K, Schoor K D, Hartl D L. (1991) Identification of nucleotide substitutions necessary for trans-activation of mariner transposable elements in Drosophila: analysis of naturally occurring elements. Genetics, 128: 777-84.
57. Massie B, Gluzman Y, Hassell J A. (1986) Construction of a helper-free recombinant adenovirus that expresses polyomavirus large T antigen. Mol Cell Biol., 6: 2872-83.
58. Meinkoth J, Wahl G. (1984) Hybridization of nucleic acids immobilized on solid supports. Anal Biochem., 138: 267-84.
59. Morsy M A, Alford E L, Bett A, Graham F L, Caskey C T. (1993) Efficient adenoviral-mediated ornithine transcarbamylase expression in deficient mouse and human hepatocytes. J Clin Invest., 92: 1580-86.
60. Moullier P, Bohl D, Heard J M, Danos O. (1993) Correction of lysosomal storage in the liver and spleen of MPS VII mice by implantation of genetically modified skin fibroblasts. Nat. Genet., 4: 154-9.
61. Mulligan R C. (1993) The basic science of gene therapy. Science, 260: 926-32.
62. Mulligan R C, Berg P. (1980) Expression of a bacterial gene in mammalian cells. Science, 209: 1422-27.
63. Murakami S, Nakashima R, Yamashita E, Yamaguchi A. (2002) Crystal structure of bacterial multidrug efflux transporter AcrB. Nature, 419: 587-93.
64. Murakami S, Tamura N, Saito A, Hirata T, Yamaguchi A. (2004) Extramembrane central pore of multidrug exporter AcrB in Escherichia coli plays an important role in drug transport. J Biol. Chem., 279: 3743-48.
65. Neale A D, Scopes R K, Kelly J M, and Wettenhall R E H. (1986). The two alcohol dehydrogenases of Zymomonas mobilis: purification by differential dye ligand chromatography, molecular characterization and physiological role. Eur. J. Biochem., 154: 119-124.
66. Neutze R, Pebay-Peyroula E, Edman K, Royant A, Navarro J, Landau E M. (2002) Bacteriorhodopsin: a high-resolution structural view of vectorial proton transport. Biochim Biophys Acta., 1565: 144-67.
67. Osborne T F, Arvidson D N, Tyau E S, Dunsworth-Browne M, Berk A J. (1984) Transcription control region within the protein-coding portion of adenovirus E1A genes. Mol Cell Biol., 4: 1293-305.
68. Osterwalder T, Yoon K S, White B H, Keshishian H. (2001). A conditional tissue-specific transgene expression system using inducible GAL4. Proc. Natl. Acad. Sci. U S A., 98: 12596-12601.
69. Persson R, Wohlfart C, Svensson U, Everitt E. (1985) Virus-receptor interaction in the adenovirus system: characterization of the positive cooperative binding of virions on HeLa cells. J. Virol., 54: 92-7.
70. Peterson C L, Hammond B, Reece D, Thompson J, and Beck S. (1995). Performance and durability testing of diesel engines using ethyl and methyl ester fuels. Report submitted in completion for contracts 236-1 and 52016-1 from the National Biodiesel Board USA. Department of Biological and Agricultural Engineering, University of Idaho, Moscow, USA.
71. Pietersz G A, McKenzie I F. (1992) Antibody conjugates for the treatment of cancer. Immunol Rev., 29: 57-80.
72. Poole K. (2004) Efflux-mediated multiresistance in Gram-negative bacteria. Clin. Microbio. Infect., 10: 12-26.
73. Pos K M, Diederichs K. (2002) Purification, crystallization and preliminary diffraction studies of AcrB, an inner-membrane multi-drug efflux protein. Acta Crystallogr. D Biol Crystallogr., 58: 1865-67.
74. Pos K M, Schiefner A, Seeger M A, Diederichs K. (2004) Crystallographic analysis of AcrB. FEBS Lett., 564: 333-9.
75. Ragot T, Finerty S, Watkins P E, Perricaudet M, Morgan A J. (1993) Replication-defective recombinant adenovirus expressing the Epstein-Barr virus (EBV) envelope glycoprotein gp340/220 induces protective immunity against EBV-induced lymphomas in the cottontop tamarin. J Gen Virol.; 74: 501-7.
76. Ram Z, Culver K W, Walbridge S, Blaese R M, Oldfield E H. (1993) In situ retroviral-mediated gene transfer for the treatment of brain tumors in rats. Cancer Res., 53: 83-8.
77. Rich D P, Couture L A, Cardoza L M, Guiggio V M, Armentano D, Espino P C, Hehir K, Welsh M J, Smith A E, Gregory R J. (1993) Development and analysis of recombinant adenoviruses for gene therapy of cystic fibrosis. Hum Gene Ther., 4: 461-76.
78. Roessler B J, Allen E D, Wilson J M, Hartman J W, Davidson B L. (1993) Adenoviral-mediated gene transfer to rabbit synovium in vivo. J Clin Invest., 92: 1085-92.
79. Roffler S R, Wang S M, Chem J W, Yeh M Y, Tung E. (1991) Anti-neoplastic glucuronide prodrug treatment of human tumor cells targeted with a monoclonal antibody-enzyme conjugate. Biochem Pharmacol., 42: 2062-65.
80. Rubin G M, Spradling A C. (1982) Genetic transformation of Drosophila with transposable element vectors. Science, 218: 348-53
81. Scopes R K (1983). An iron-activated alcohol dehydrogenase. FEBS Lett., 156: 303-306.
82. Seeger M A, Schiefner A, Eicher T, Verrey F, Diederichs K, Pos K M. (2006) Structural asymmetry of AcrB trimer suggests a peristaltic pump mechanism. Science, 313: 1295-8.
83. Senter P D, Su P C, Katsuragi T, Sakai T, Cosand W L, Hellstrom I, Hellström K E.
(1991) Generation of 5-fluorouracil from 5-fluorocytosine by monoclonal antibody-cytosine deaminase conjugates. Bioconjug Chem., 2: 447-51.
84. Senter P D, Wallace P M, Svensson R P, Vrudhula V M, Kerr D E, Hellstrom I, Hellström K E. (1993) Generation of cytotoxic agents by targeted enzymes. Bioconjug Chem., 4: 3-9.
85. Seth P, Fitzgerald D, Ginsberg H, Willingham M, Pastan I. (1984) Evidence that the penton base of adenovirus is involved in potentiation of toxicity of Pseudomonas exotoxin conjugated to epidermal growth factor. Mol Cell Biol., 4: 1528-33.
86. Seth P, Fitzgerald D J, Willingham M C, Pastan I. (1984) Role of a low-pH environment in adenovirus enhancement of the toxicity of a Pseudomonas exotoxin-epidermal growth factor conjugate. J. Virol., 51: 650-5.
87. Slotboom D J, Sobczak I, Konings W N, Lolkema J S. (1999) A conserved serine-rich stretch in the glutamate transporter family forms a substrate-sensitive reentrant loop. Proc Natl Acad Sci USA., 96: 14282-87.
88. Sobczak I, Lolkema J S. (2005) Structural and mechanistic diversity of secondary transporters. Curr Opin Microbiol., 8: 161-7.
89. Southern P J, Berg P. (1982) Transformation of mammalian cells to antibiotic resistance with a bacterial gene under the control of the SV40 early region promoter. Journal of Molecular and Applied Genetics, 1: 327-41.
90. Spradling A C. P element-mediated transformation. In: Drosophila—A Practical Approach (Roberts, D. B., ed.), pp. 175-197, IRL Press, Oxford (1986).
91. Spradling A C, Rubin G M. (1982) Transposition of cloned P elements into Drosophila germ line chromosomes. Science, 218: 341-7.
92. Steller H, Pirrotta V. (1986) P transposons controlled by the heat shock promoter. Mol Cell Biol., 6: 1640-9.
93. Sugden B, Marsh K, Yates J. (1985) A vector that replicates as a plasmid and can be efficiently selected in B-lymphoblasts transformed by Epstein-Barr virus. Mol Cell Biol., 5: 410-3.
94. Sun T Q, Fernstermacher D A, Vos J M. (1994) Human artificial episomal chromosomes for cloning large DNA fragments in human cells. Nat. Genet., 8: 33-41.
95. Tamura N, Murakami S, Oyama Y, Ishiguro M, Yamaguchi A. (2005) Direct interaction of multidrug efflux transporter AcrB and outer membrane channel TolC detected via site-directed disulfide cross-linking. Biochemistry, 44: 11115-21.
96. Toyoshima C, Inesi G. (2004) Structural basis of ion pumping by Ca2+-ATPase of the sarcoplasmic reticulum. Annu Rev Biochem., 73: 269-92.
97. van der Putten H, Botteri F M, Miller A D, Rosenfeld M G, Fan H, Evans R M, Verma I M. (1985) Efficient insertion of genes into the mouse germ line via retroviral vectors. Proc Natl Acad Sci USA, 82: 6148-52.
98. Vaneechoutte M, Young D M, Ornston L N, De Baere T, Nemec A, Van Der Reijden T, Can E, Tjernberg I, Dijkshoom L. (2006) Naturally transformable Acinetobacter sp. strain ADP1 belongs to the newly described species Acinetobacter baylyi. Appl Environ Microbiol., 72: 932-6.
99. Varga M J, Weibull C, Everitt E. (1991) Infectious entry pathway of adenovirus type 2. J. Virol., 65: 6061-70.
100. Wickham T J, Mathias P, Cheresh D A, Nemerow G R. (1993) Integrins alpha v beta 3 and alpha v beta 5 promote adenovirus internalization but not virus attachment. Cell, 73: 309-19.
101. Wolff J A, Malone R W, Williams P, Chong W, Acsadi G, Jani A, Feigner P L. (1990) Direct gene transfer into mouse muscle in vivo. Science, 247: 1465-68.
102. Yernool D, Boudker O, Jin Y, Gouaux E. (2004) Structure of a glutamate transporter homologue from Pyrococcus horikoshii. Nature, 431: 811-8.
103. Yu E W, McDermott G, Zgurskaya H I, Nikaido H, Koshland D E Jr. (2003) Structural basis of multiple drug-binding capacity of the AcrB multidrug efflux pump. Science, 300: 976-80.
104. Zabner J, Couture L A, Gregory R J, Graham S M, Smith A E, Welsh M J. (1993) Adenovirus-mediated gene transfer transiently corrects the chloride transport defect in nasal epithelia of patients with cystic fibrosis. Cell, 75: 207-16.
105. Zabner J, Petersen D M, Puga A P, Graham S M, Couture L A, Keyes L D, Lukason M J, St George J A, Gregory R J, Smith A E, et al. (1994) Safety and efficacy of repetitive adenovirus-mediated transfer of CFTR cDNA to airway epithelia of primates and cotton rats. Nat. Genet., 6: 75-83.
106. Zhang W W, Fang X, Branch C D, Mazur W, French B A, Roth J A. (1993) Generation and identification of recombinant adenovirus by liposome-mediated transfection and PCR analysis. Biotechniques, 15: 868-72.
107. U.S. Pat. No. 4,413,058, Continuous production of ethanol by use of flocculent zymomonas mobilis, issued on Nov. 1, 1983 to Edward J. Arcuri and Terrence L. Donaldson.
108. U.S. Pat. No. 4,242,455, Process for the acid hydrolysis of carbohydrate polymers and the continuous fermentation of the sugars obtained therefrom to provide ethanol, issued on Dec. 30, 1980 to Werner C. Muller and Franklyn D. Miller.
109. U.S. Pat. No. 4,350,765, Method for producing ethanol with immobilized microorganism, issued on Sep. 21, 1982 to Ichiro Chibata, Jyoji Kato, and Mitsuru Wada.
110. U.S. Pat. No. 4,670,388, Method of incorporating DNA into genome of drosophila, issued on Jun. 2, 1987 to Gerald M. Rubin and Allan C. Spradling.
111. U.S. Pat. No. 4,868,116, Introduction and expression of foreign genetic material in epithelial cells, issued on Sep. 19, 1989 to Jeffrey R. Morgan and Richard C. Mulligan.
112. U.S. Pat. No. 4,897,355, N[W,(W-1)-dialkyoxy]- AND N[W,(W-1)-dialkenyloxy]-ALK-1-YL-N,N,N-tetrasubstituted ammonium lipids and uses therefore, issued on Jan. 30, 1990 to Deborah A. Eppstein, Philip L. Feigner, Thomas R. Gadek, Gordon H. Jones, Richard B. Roman.
113. U.S. Pat. No. 4,980,286, In vivo introduction and expression of foreign genetic material in epithelial cells, issued on Dec. 25, 1990 to Jeffrey R. Morgan and Richard C. Mulligan.
114. PCT/GB1988/000411, Thermophilic ethanol production, filed on May 24, 1988, by Brian S. Hartley.
115. PCT/US1989/000422, Modified hepatocytes and uses therefore, filed on Feb. 2, 1989, by James M. Wilson and Richard C. Mulligan.
116. PCT/US 1989/003794, Recombinant retroviruses with amphotropic and ecotropic host ranges, filed on Sep. 1, 1989, by Olivier Danos and Richard C. Mulligan.
117. PCT/JP2006/324501, Acyl-CoA: ethanol O-acyltransferase/esterase gene and uses thereof, filed on Dec. 1, 2006, by Yoshihiro Nakao, Yukiko Kodama, and Tomoko Shimonaga.

What is claimed is:

1.-27. (canceled)

28. A method of excreting a product from a cell, wherein the cell comprises a transmembrane pump, the method comprising producing a product in the cell, wherein the product is excreted from the cell into the medium via the transmembrane pump.

29. The method of claim 28, wherein the transmembrane pump is a multidrug efflux pump.

30. The method of claim 29, wherein the multidrug efflux pump is a resistance-nodulation-division (RND) transporter.

31. The method of claim 30, wherein the expression of the resistance-nodulation-division transporter is overexpressed.

32. The method of claim 28, wherein the product is selected from the group consisting of antibiotics, amphipathic pharmaceuticals, hydrophobic dyes, hydrophobic pigments, hydrophobic fragrances, hydrophobic flavors, waxy products, monomers and oligomers for polymer assembly, vegetable oils, hydrophobic vitamins and vitamin precursors, hormones, lipids, and other biologically active molecules.

33. The method of claim 28, wherein the cell is an algae cell.

34. The method of claim 33, wherein the algae cell is photosynthetic.

35.-37. (canceled)

38. The method of claim 28, further comprising harvesting the excreted product.

39. The method of claim 38, wherein harvesting the excreted product further comprises skimming.

40. The method of claim 38, wherein harvesting the excreted product occurs without killing the cell.

41. The method of claim 28, wherein producing the excreted product is continuous.

42. The method of claim 28, wherein the cell further comprises at least one transgenic insert.

43. The method of claim 42, wherein the at least one transgenic insert comprises Acyl-CoA:ethanol acyltransferase.

44. The method of claim 43, wherein Acyl-CoA:ethanol acyltransferase comprises SEQ ID NO: 1 or SEQ ID NO: 2.

45. The method of claim 43, wherein the at least one transgenic insert further comprises pyruvate decarboxylase, alcohol dehydrogenase, or both.

46. The method of claim 43, wherein the expression of Acyl-CoA:ethanol acyltransferase comprises is overexpressed.

47. The method of claim 45, wherein the expression of pyruvate decarboxylase, alcohol dehydrogenase, or both is overexprssed.

48. The method of claim 32, wherein the product is a fatty acid ethyl ester.

49. The method of claim 48, wherein the diffusivity of the fatty acid ethyl ester is enhanced.

50. The method of claim 48, wherein the fatty acid ethyl ester is a biodiesel component.
